(19) United States
(12) Patent Application Publication
(10) Pub. No.: US 2023/0173061 A1
(43) Pub. Date: Jun. 8, 2023

(54) HUMAN CYTOMEGALOVIRUS POLYEPITOPE VACCINE COMPOSITION

(71) Applicant: The Council of the Queensland Institute of Medical Research, 300 Herston Road (AU)

(72) Inventors: Rajiv Khana, Herston, Queensland (AU); Vijayendra Dasari, Herston, Queensland (AU)

(73) Assignee: The Council of the Queensland Institute of Medical Research, Herston, Queensland (AU)

(21) Appl. No.: 17/921,945

(22) PCT Filed: Apr. 28, 2021
(86) PCT No.: PCT/AU2021/050385

§ 371 Date: Oct. 27, 2022

(30) Foreign Application Priority Data
Apr. 28, 2020 (AU) 2020901334

(51) Int. Cl. A61K 39/245; C07K 14/045; A61P 31/22

(52) U.S. Cl. CPC A61K 39/245; C07K 14/045; A61P 31/22; A61K 2039/55561

(57) ABSTRACT

Disclosed is a human herpesvirus immunotherapy. More particularly, disclosed is a composition that includes one or more recombinant proteins that include a plurality of epitopes derived from multiple human cytomegalovirus antigens, a CMV envelope5glycoprotein, and a TLR agonist.

### RELATED APPLICATIONS

This application claims priority to Australian Provisional Application No. 2020901334 entitled “Pharmaceutical Composition”, filed on 28 Apr. 2020, the entire content of which is hereby incorporated herein by reference in its entirety.

### TECHNICAL FIELD

This invention relates to human herpesvirus immunotherapy. In particular, the invention relates to a vaccine composition that includes one or more recombinant proteins that include a plurality of epitopes derived from multiple human cytomegalovirus (CMV) antigens, a CMV envelope glycoprotein, and a TLR agonist, which, when used in immunotherapy is capable of eliciting a protective and durable humoral and cell mediated immune response, without being limited thereto.

### BACKGROUND OF THE INVENTION

Primary CMV in healthy individuals is generally asymptomatic, establishing a latent state with occasional reactivation and shedding from mucosal surfaces. In some cases, primary CMV infection is accompanied with clinical symptoms of a mononucleosis-like illness, similar to that caused by Epstein-Barr virus. There are two important clinical settings where CMV causes significant morbidity and mortality. These include congenital primary infection and primary or reactivation of virus in immunosuppressed adults.

Recent understandings in the immunology, pathology, and molecular biology of CMV have suggested that protection against CMV-related disease is mediated by both humoral and cellular immunity, thus, an ideal vaccine against CMV needs to induce both humoral and cellular responses. Unfortunately, recent attempts to develop a CMV vaccine have demonstrated limited success. These CMV vaccine strategies have assessed glycoprotein B (gB), pp65 and IE-1 as potential targets and they have been delivered by numerous delivery platforms, including the attenuated CMV Towne strain (Jacobson, Sinclair et al. 2006) recombinant viral vectors encoding full length antigens and epitopes (Bernstein, Reap et al. 2009, Zhong and Khanna 2009, La Rosa, Longmate et al. 2017), DNA (Wloch, Smith et al. 2008) dense body (Pepperl-Klindworth, Frankenberg et al. 2002), subunit (Drulak, Malinoski et al. 2000) vaccines and most recently a conditionally replication-defective CMV vaccine derived from AD169 strain (Adler, Lewis et al. 2019). Over the years it has been believed that in order to elicit a protective, CD8 cytotoxic T cell response, viral antigens must be delivered in nucleic acid form (e.g., using a viral vector delivery system or DNA plasmids) rather than as exogenously-delivered proteins, so that the expressed polypeptide is properly processed and presented to T cells. However, the majority of these vaccine delivery platforms, in particular live-attenuated vaccines and viral vector based vaccines, have raised several regulatory concerns, such as perceived long-term theoretical health risks and pre-existing immunity (Lee, Markham et al. 2012).

Accordingly, there remains a need for a vaccine formulation for the prevention of CMV infection and reactivation that overcomes one or more of the deficiencies of prior art vaccines, including immunogenicity and safety issues thereof.

### SUMMARY

The present invention addresses a need for the development of herpesvirus, and in particular CMV, immunotherapy using a safe delivery technology that is capable of eliciting a durable and protective immune response. The invention is directed towards reducing the risk of CMV associated injury to the developing foetus, and immunologically compromised individuals such as recipients of solid organ and hematopoietic stem cell transplants and patients with advanced HIV disease.

The invention has surprisingly arisen from the discovery that a vaccine composition that includes a polyepitope protein containing multiple human CMV antigens, a CMV envelope glycoprotein and a CpG oligonucleotide that activates toll-like receptor administered to an individual may elicit a protective humoral and cell-mediated immune response that is durable.

Accordingly, the invention is broadly directed to a pharmaceutical composition comprising one or more isolated proteins that include a plurality of epitopes, such as cytotoxic T-lymphocyte (CTL) epitopes, from two or more different CMV antigens, a CMV envelope protein and a TLR agonist and methods of using same.

In a first aspect, the invention relates to a pharmaceutical composition comprising:

(a) one or a plurality of isolated proteins comprising a plurality of epitopes, wherein the plurality of epitopes are derived from two or more different CMV antigens;

(b) a CMV envelope protein, or a fragment, variant or derivative thereof; and

(c) a TLR9 agonist.

In one embodiment, the composition is capable of inducing or eliciting a humoral immune response and a cell-mediated immune response, such as a cytotoxic T-lymphocyte immune response, upon administration to a subject. In this regard, one or more of the plurality of epitopes may be or comprise a CTL epitope.

Suitably, the one or plurality of isolated proteins is or comprises a polytope protein comprising two or more of the plurality of epitopes derived from the two or more different CMV antigens. The polytope protein may further comprise intervening amino acids or amino acid sequences. In one embodiment, the polytope protein comprises an intervening amino acid or amino acid sequence between at least two of said epitopes comprising proteasome liberation amino acids or amino acid sequences, such as AD, K and/or R.

Suitably, the epitopes are selected to provide broad coverage of the human population. In certain embodiments, the epitopes are restricted by HLA class I specificities HLA-A1, -A2, -A3, -A11, -A23, -A24, -A26, -A29, -A30, -B7, -B8, -B18, -B27, -B35, -B38, -B40, -B41, -B44, -B51, -B57, -B58 and/or -CW6. More particularly, the epitopes may be restricted by HLA class I specificities HLA-A1, -A2, -A3, -A11, -A23, -A24, -B7, -B8, -B18, -B27, -B35, -B40, -B44, -B57, -B58 and/or -CW6.

Suitably, the epitopes are selected to target one or more stages of CMV infection, such as infection, replication and/or packaging. In one embodiment, the epitopes are derived from pp50, pp65, pp 150, DNAse and/or IE-1. More particularly, the epitopes can have an amino acid sequence selected from those set forth in Table 1 (i.e., SEQ ID NOS: 1-20), a fragment, variant or derivative thereof or any combination thereof. In one specific embodiment, the one or plurality of isolated proteins comprise each of the epitope amino acid sequences set forth in Table 1 (i.e., SEQ ID NOS: 1-20). In this regard, the one or plurality of isolated proteins can suitably comprise an amino acid sequence set forth in SEQ ID NO:21 or a fragment, variant or derivative thereof.

TABLE 1
CMVPOLY20PL-NH Epitope Sequences
and HLA Restriction

| Epitope Sequence | HLA restriction | HCMV antigen |
|---|---|---|
| FPTKDVALAD* | HLA B35 | pp65 |
| GP1SHGHVLKAD | HLA A11 | pp65 |
| QYDPVAALFAD | HLA A24 | pp65 |
| YSEHPTFTSOYAD | HLA A1 | pp65 |
| TPRVTGGGAMR | HLA B7 | pp65 |
| QIKVRVDMVR | HLA B8 | IE-1 |
| IPSINVHHYR | HLA B35 | pp65 |
| TTVYPPSSTAKAD | HLA A3 | pp50 |
| RPHERNGFTVLR | HLA B7 | pp65 |
| DELRRKMMYMAD | B8/B18/B44 | IE-1 |
| VTEHDTLLYK | HLA A1 | pp50 |
| NLVPMVATVK | HLA A2 | pp65 |
| VLEETSVMLK | HLA A2 | IE-1 |
| AYAOKIFKILAD | A23/A24 | pp65 |
| TRATKMQVIAD | CW6 | pp65 |
| ARVYEIKCRR | HLA B27 | DNAse |
| KEVNSQLSLK | HLA B40 | IE-1 |
| ELKRKMIYMK | HLA B8 | IE-1 |
| YILEETSVMLK | HLA A2 | IE-1 |
| QAIRETVELK | B57/B58 | pp65 |

Underlined amino acids show proteasome liberation sequences

In particular embodiments, the one or plurality of isolated proteins comprise twenty (20) or less epitopes.

Suitably, the CMV envelope protein is selected from the group consisting of glycoprotein B (gB), glycoprotein H (gH), glycoprotein L (gL), glycoprotein M (gM), glycoprotein N (gN), glycoprotein O (gO), variants, fragments or derivatives thereof and any combination thereof. In one specific embodiment, the CMV envelope protein is or comprises CMV glycoprotein B, or a fragment, variant or derivative thereof.

Suitably, the TLR9 agonist comprises CpG ODN1018 and/or CpG ODN2006. In one particular embodiment, the TLR9 agonist is or comprises CpG ODN1018. In some embodiments, the TLR9 agonist is not MPL, CpG ODN1826, CpG ODN2006, CpG ODN2216 and/or CpG ODN2336.

Suitably, the pharmaceutical composition of the present aspect further comprises a pharmaceutically-acceptable carrier, diluent or excipient.

In some embodiments, the pharmaceutical composition further comprises one or more immunostimulatory molecules or adjuvants, such as in addition to the TLR9 agonist.

Suitably, the pharmaceutical composition of the present aspect is an immunogenic composition suitable for use in the prophylactic or therapeutic treatment or prevention of a disease, disorder or condition associated with a CMV infection in a subject.

In a second aspect, the invention provides a vaccine comprising the pharmaceutical composition of the first aspect for eliciting a protective immune response against CMV or a CMV infection.

In a third aspect, the invention resides a method of treating or preventing a CMV infection in a subject, said method including the step of administering to the subject a therapeutically effective amount of a pharmaceutical composition comprising:

(a) one or a plurality of isolated proteins comprising a plurality of epitopes, wherein the plurality of epitopes are derived from two or more different CMV antigens;

(b) a CMV envelope protein, or a fragment, variant or derivative thereof; and

(c) a TLR9 agonist;

to thereby prevent or treat the CMV infection in the subject.

In a fourth aspect, the invention provides a method of eliciting an immune response to a CMV antigen in a subject, said method including the step of administering to the subject a therapeutically effective amount of a pharmaceutical composition comprising:

(a) one or a plurality of isolated proteins comprising a plurality of epitopes, wherein the plurality of epitopes are derived from two or more different CMV antigens;

(b) a CMV envelope protein, or a fragment, variant or derivative thereof; and

(c) a TLR9 agonist;

to thereby elicit the immune response in said subject.

In one embodiment, the immune response is or comprises a humoral immune response and/or a cell-mediated immune response, such as a cytotoxic T-lymphocyte immune response.

The method of the present aspect suitably elicits a protective immune response against CMV or a CMV infection in the subject.

In a fifth aspect, the invention relates to a method of inducing immunity against a CMV infection in a subject, said method including the step of administering to the subject a therapeutically effective amount of a pharmaceutical composition comprising:

(a) one or a plurality of isolated proteins comprising a plurality of epitopes, wherein the plurality of epitopes are derived from two or more different CMV antigens;

(b) a CMV envelope protein, or a fragment, variant or derivative thereof; and

(c) a TLR9 agonist;

to thereby induce immunity against the CMV infection in the subject.

The method of the present aspect suitably induces a protective immune response against the CMV infection.

In another embodiment, the present method suitably induces a humoral immune response and/or a cell-mediated immune response, such as a cytotoxic T-lymphocyte immune response, against the CMV infection.

Suitably, according to the three aforementioned aspects the subject is a mammal. More preferably, the subject is a human.

Referring to the method of the third, fourth and fifth aspects, the pharmaceutical composition is suitably that of the first aspect.

In a sixth aspect, the invention resides in an isolated protein comprising each of the epitope amino acid sequences set forth in SEQ ID NOS: 1-20 or a fragment, variant or derivative thereof.

Suitably, the isolated protein comprises, consists essentially of, or consists of the amino acid sequence set forth in SEQ ID NO:21 or a fragment, variant or derivative thereof.

In particular embodiments, the isolated protein is for use in the method of the three aforementioned aspects.

In a seventh aspect, the invention provides an isolated nucleic acid encoding the isolated protein of sixth aspect.

In an eighth aspect, the invention resides in a genetic construct comprising the isolated nucleic acid of the seventh aspect.

In a ninth aspect, the invention relates to a host cell comprising the isolated nucleic acid of the seventh aspect and/or the genetic construct of the eighth aspect.

In a tenth aspect, the invention provides a method of producing the isolated protein of the sixth aspect, including the steps of; (i) culturing the host cell of ninth aspect; and (ii) isolating said isolated protein from said host cell cultured in step (i).

As used herein, the indefinite articles ‘a’ and ‘an’ are used here to refer to or encompass singular or plural elements or features and should not be taken as meaning or defining “one” or a “single” element or feature. For example, “a” protein includes one protein, one or more proteins or a plurality of proteins.

Unless the context requires otherwise, the terms “comprise”, “comprises” and “comprising”, or similar terms are intended to mean a non-exclusive inclusion, such that a recited list of elements or features does not include those stated or listed elements solely, but may include other elements or features that are not listed or stated.

By “consisting essentially of” in the context of an amino acid sequence, such as an isolated protein, is meant the recited amino acid sequence together with an additional one, two or three amino acids at the N- or C-terminus.

### BRIEF DESCRIPTION OF THE FIGURES

In order that the present invention may be more readily understood and placed into practical effect, preferred embodiments of the invention will be described, by way of example only, with reference to the accompanying figures.

FIG. 1: Nucleotide and amino acid sequence encoding CMVpoly20PL-NH protein

FIG. 2: An SDS-PAGE gel analysis of the CMVpoly20PL-NH protein expression, cell lysis, inclusion bodies wash and protein purification: DNA sequence encoding CMVpoly20PL-NH was cloned into an IPTG inducible plasmid, pJexpress 404 and transformed into chemically competent E. coli BL21-codonPlus (DE3) RP host for protein expression. CMVpoly20PL-NH protein expression was carried out by adding 1 mM/mL of IPTG for 4 hours. After 4 hours of protein induction expression levels were determined by analysing un-induced (ui) and induced (ind) samples on the SDS-PAGE (A). To determine the solubility of CMVpoly20PL-NH, cells were lysed, supernatant and pellet fractions were analyzed on SDS-PAGE gel (B). Since protein was present in inclusion bodies (IBs), they were washed with TE buffer and then with a buffer containing 100 mM NaH2PO4, 10 mM Tris, 4M urea pH 7.5 three times (W1 to W3), protein was solubilized in 100 mM NaH2PO4, 10 mM Tris, 2.5 mM DTT, 8M urea pH 5.5 buffer and then supernatant (sup) and pellet fractions were analysed on SDS-PAGE (C). Following IBs solubilisation, CMVpoly20PL-NH protein was purified using SP-Sepharose and Q-Sepharose chromatography techniques and the purified protein was dialysed against 25 mM glycine pH 3.8 buffer and passed through Mustang E membrane (D & E).

FIG. 3: Protein intact mass analysis of CMVpoly20PL-NH: Protein samples were diluted to a concentration approximately 0.25 ug/uL with 2% acetonitrile and 0.1% formic acid. Sample (5 μL) was injected onto the column. Proteins was desalted on the column and was eluted from the column using a linear solvent gradient (A: 99.9% water+0.1% formic acid; B:99.9% acetonitrile+0.1% formic acid). The eluent was subject to positive ion electrospray MS analysis on Q Exactive which was scanned from 600 m/z to 3800 m/z, at 17,500 resolution. The protein ESI spectra were averaged over the protein liquid chromatography elution profile. The multiply charged protein spectrum was de-convoluted by Thermo Protein Deconvolution 2.0 software.

FIG. 4: CMVpoly20PL-NH in vitro immunogenicity assessment: To determine the immunogenicity of CMVpoly protein T1 (HLA A2) cells in log phase culture were washed with RPMI medium containing no serum and then pulsed with 25 μg of CMVpoly20PL-NH protein for one hour. CMVpoly20PL-NH pulsed T1 cell were washed and then incubated overnight in RPMI containing 10% FCS. Following overnight incubation cell were washed and exposed to CMV HLA A2 restricted epitope NLVPMVATV(NLV)-specific CD8+ T cells at 1 CD8+ T cell: 1APC and 4 CD8+ T cells: 1APC ratios and incubated for four hours. Intracellular IFN-g expression was determined by ICS analysis. T1 cells pulsed with and without NLV peptide used as positive and negative controls.

FIG. 5: Immunogenicity evaluation of CMV vaccine formulated with CpG 1018 alone or with various forms MPLA in human HLA A24 transgenic mice: CMV vaccine formulations were prepared by mixing CMV gB (5 μg) and CMVpoly20PL-NH (30 μg) proteins with PHAD (25 μg) and CpG 1018 (50 μg) (G1V), 3D-PHAD (25 μg) and CpG 1018 (50 μg) (G2V), 3D(6-acyl)-PHAD (25 μg) and CpG 1018 (50 μg) (G3V) or CpG 1018 (50 μg) alone (G4V). As a positive control we formulated CMV gB (5 μg) and CMVpoly20PL-NH (30 μg) proteins with MPL (25 μg) and CpG 1826 (50 μg) (G5V) and as a negative controls we formulated adjuvants alone (G6C to G10C). Human HLA A24 transgenic mice were immunised subcutaneously on day 0, boosted on day 21 with an identical vaccine formulation (V) or adjuvants alone (C) and then sacrificed on day 28 to assess the CMV-specific CD4+ and CD8+ T cells responses using ICS assay and humoral immune responses using ELISA (A). On day 28 splenocytes were prepared and then stimulated with respective CMV CD8+ T cell peptides (HLA A24 restricted peptides-QYD & AYA) or with CMV gB pepmix consisting of a pool of 224 peptides (15mers with 11 aa overlap) in the presence of brefeldin A and the CMV-specific CD4+T and CD8+ T cells producing IFN-γ was measured using an ICS assay. (B) shows ex vivo percentage of CMV-specific CD8+ T cell producing IFN-γ following stimulation with CMV CD8+ T cell peptides in HLA A24 transgenic mice. On day 28 splenocytes from vaccinated and control mice were stimulated with HLA A24 restricted peptides and then cultured for 10 days. On day 10 T cell specificity was assessed using ICS assay. (C) shows percentage of CMV-specific CD8+ T cells producing IFN-γ following in vitro stimulation with CMV HLA A24 restricted CD8+ T cell peptides and (D) shows expression of IFN-γ, TNF-α, CD107a and IL-2 cytokines by in vitro expanded CMV-specific CD8+ T cells from HLA A24 mice immunized with CMV vaccine formulation. (E and F) shows ex vivo percentage of CMV gB-specific CD4+ T and CD8+ T cells producing IFN-γ following immunisation with CMV vaccine or adjuvant alone. Error bars represent the mean±SEM *, P<0.05; **, P<0.01; ***, P<0.001, ns=not significant (determined by the student t test).

FIG. 6: Assessment of CMV-specific humoral immune responses in human HLA A24 transgenic mic.: Human HLA A24 transgenic mice were vaccinated with CMV gB (5 μg) and CMVpoly20PL-NH (30 μg) proteins with PHAD (25 μg) and CpG 1018 (50 μg) (G1V), 3D-PHAD (25 μg) and CpG 1018 (50 μg) (G2V), 3D(6-acyl)-PHAD (25 μg) and CpG 1018 (50 μg) (G3V), CpG 1018 (50 μg) alone (G4V), MPL (25 μg) and CpG 1826 (50 μg) (G5V) or adjuvants alone (G6C to G10C) on day 0, mice were tail bled on day 21 and a booster dose was given. Mice were then sacrificed in day 28 to assess humoral immune responses using ELISA. (A and B) shows CMV gB-specific antibody titres in serum samples of A24 transgenic mice on day 21 (primary immunization) and 28 (boost) and (C) shows prevalence of CMV gB-specific antibody isotypes (IgM, IgA, IgG1, IgG2a, IgG2b and IgG3) on day 21 and day 28 in pooled serum samples obtained from HLA A24 mice.

FIG. 7: Evaluation of CMV-specific CD8+ T cell responses following vaccination of multiple HLA transgenic mice with CMV gB, CMVpoly20PL-NH and CpG 1018 vaccine formulation. Multiple Human HLA transgenic mice (HLA A1, HLA 2, HLA A24, HLA B8 and HLA B35) were immunized with CMV gB (5 μg) and CMVpoly20PL-NH (30 μg) formulated with CpG 1018 (50 μg) on day 0. On day 21 mice were tail bled and boosted with identical vaccine formulation. On day 28 Mice were sacrificed to assess CMV-specific CD8+ T cells immune responses in multiple human HLA transgenic mice (A). On day 28 mice were sacrificed and splenocytes were prepared and then stimulated with respective CMV CD8+ T cell peptides (HLA A1-VTE & YSE; HLA A2-NLV, VLE & YIL; HLA A24-QYD & AYA; HLA B8-QIK, ELR & ELK; HLA B35-FPT & IPS) in the presence of brefeldin A and the CMV-specific CD8+ T cells producing IFN-γ was measured using an ICS assay. (B) shows ex vivo percentage of CMV-specific CD8+ T cells producing IFN-γ following stimulation with CMV CD8+ T cell peptides. Evaluating CMV-specific CD8+ T cell responses in in vitro stimulated splenocytes. Following immunisation splenocytes were in vitro stimulated with respective CMV CD8+ T cell peptide and cells were cultured for 10 days. On day 10, T cell specificity was assessed using ICS assay. (C) shows percentage of CMV-specific CD8+ T cells producing IFN-γ following in vitro stimulation with CMV CD8+ T cell peptides. (D) shows representative FACS plots of the frequency of IFN-γ producing CMV-specific CD8+ T cells (ex vivo and in vitro expanded) from one HLA transgenic mouse. (E) shows expression of IFN-γ, TNF-α, CD107a and IL-2 cytokines by ex vivo and in vitro expanded CMV-specific CD8+ T cells from HLA transgenic mice immunized with CMV gB, CMVpoly20PL-NH and CpG 1018 vaccine formulation. Error bars represent the mean±SEM. *, P<0.05; **, P<0.01; ***, P<0.001, ns=not significant (determined by the student t test).

FIG. 8: Evaluation of CMV gB-specific CD4+ T cell responses following vaccination of multiple HLA transgenic mice with CMV gB, CMVpoly20PL-NH and CpG 1018 vaccine formulation. Human HLA transgenic mice (HLA A1, HLA 2, HLA A24, HLA B8 and HLA B35) were immunized with CMV gB (5 μg) and CMVpoly20PL-NH (30 μg) formulated with CpG 1018 (50 μg) on day 0. On day 21 mice were tail bled and boosted with an identical vaccine formulation. On day 28 Mice were sacrificed to assess CMV gB-specific CD4+ and CD8+ T cell immune responses in multiple human HLA transgenic mice (A). On day 28 mice were sacrificed and splenocytes were prepared and then stimulated with CMV gB pepmix consisting of a pool of 224 peptides (15mers with 11 aa overlap) in the presence of brefeldin A and the CMV gB-specific CD4+ T cells producing IFN-γ was measured using an ICS assay. (A) shows ex vivo percentage of CD4+ T cell producing IFN-γ following stimulation with CMV gB pepmix in various HLA transgenic mice. Evaluating CMV gB-specific CD4+ and CD8+ T cell responses in in vitro stimulated splenocytes. Following immunisation splenocytes were stimulated in vitro with CMV gB pepmix consisting of a pool of 224 peptides (15mers with 11 aa overlap) and cells were cultured for 10 days. On day 10, T cell specificity was assessed using ICS assay. (B) shows percentage of CMV gB-specific CD4+ T cells producing IFN-γ following in vitro stimulation with CMV gB pepmix. (C) shows representative FACS plots of the frequency of IFN-γ producing CMV gB-specific CD4+ T cells (ex vivo and in vitro expanded) from one HLA transgenic mouse. (D) shows expression of IFN-γ, TNF-α, CD107a and IL-2 cytokines by ex vivo and in vitro expanded CMV gB-specific CD4+ T cells. (E) shows expression of IFNγ alone (bar graph) or IFN-γ, TNF-α, CD107a and IL-2 cytokines (pie chart) or FACS plots of in vitro expanded CMV gB-specific CD8+ T cells from all HLA transgenic mice immunised with CMV gB, CMVpoly20PL-NH and CpG 1018 vaccine formulation. Error bars represent the mean±SEM *, P<0.05; **, P<0.01; ***, P<0.001, ns=not significant (determined by the student t test).

FIG. 9: Assessment of CMV-specific humoral immune responses in multiple human HLA transgenic mice. Human HLA transgenic mice (HLA A1, HLA 2, HLA A24, HLA B8 and HLA B35) were immunized with CMV gB (5 μg) and CMVpoly20PLNH (30 μg) formulated with CpG 1018 (50 μg) on day 0. On day 21 mice were tail bled and boosted with identical vaccine formulation. On day 28 Mice were sacrificed to assess CMV gB-specific humoral immune responses using ELISA. (A and B) shows CMV gB-specific antibody titres in serum samples of all human HLA transgenic mice on day 21 (primary immunization) and prevalence of CMV gB-specific antibody isotypes (IgM, IgA, IgG1, IgG2a, IgG2b and IgG3) in pooled serum samples. (C and D) shows CMV gB-specific antibody titres in serum samples of all human HLA transgenic mice on day 28 and prevalence of CMV gB-specific antibody isotypes (IgM, IgA, IgG1, IgG2a, IgG2b and IgG3) in pooled serum samples. (E and F) shows 50% neutralizing antibody titres induced following immunization of human HLA transgenic mice to neutralise AD 169 infection of fibroblasts and TB40/E infection of adult retinal pigment epithelial cells (ARPE-19). On day 28, sera from individual groups were pooled, serially diluted, and pre-incubated with CMV AD169 or TB40/E strains. MRC-5 or ARPE 19 cells were infected with serum-treated virus and virus infectivity was determined using an IE-1/IE-2 micro-neutralization assay. Error bars represent the mean±SEM.

FIG. 10: Assessment of long-term durable CMV specific-immunity induced by CMV gB, CMVpoly20PL-NH and CpG 1018 vaccine formulation in HLA A24 transgenic mice. Human HLA A24 transgenic mice were immunized with CMV gB (5 μg) and CMVpoly20PL-NH (30 μg) formulated with CpG 1018 (50 μg) on day 0. On day 21 mice were tail bled and boosted with identical vaccine formulation on day 28 and 42 and 210. Mice were sacrificed on day 28, 42, 49, 84, 133, 203 and 217 to perform longitudinal analysis to assess CMV-specific T cell and humoral immune responses.

FIG. 11: Assessment of long-term durable CMV specific-CD8+ T cell immunity induced by CMV gB, CMVpoly20PL-NH and CpG 1018 vaccine formulation in HLA A24 transgenic mice: Human HLA A24 transgenic mice were immunized with CMV gB (5 μg) and CMVpoly20PL-NH (30 μg) formulated with CpG 1018 (50 μg) on day 0. On day 21 and 42 mice were tail bled and boosted with identical vaccine formulation on day 21 and 42 and 210. Mice were sacrificed on day 28, 42, 49, 84, 133, 203 and 217 to perform longitudinal analysis to assess CMV-specific CD8+ T cells immune responses in human HLA A24 transgenic mice. On the day of sacrifice splenocytes were prepared and then stimulated with respective CMV CD8+ T cell peptides (HLA A24-QYD & AYA) in the presence of brefeldin A and the CMV-specific CD8+ T cells producing IFN-γ was measured using an ICS assay. Evaluating CMV-specific CD8+ T cell responses in in vitro stimulated splenocytes. Following immunization splenocytes were in vitro stimulated with respective CMV CD8+ T cell peptides (HLA A24-QYD & AYA) and cells were cultured for 10 days. On day 10, T cell specificity was assessed using ICS assay. (A and B) shows percentage of CMV-specific CD8+ T cells producing IFN-γ following ex vivo and in vitro stimulation with CMV CD8+ T cell peptides. (C) shows simplified presentation of incredibly complex evaluations (SPICE) analysis of CMV-specific CD8+ T cells producing proportion of the responses comprised of various combinations of IFNγ⁺/TNF⁺/IL2⁺ cytokines. Arc indicates the proportion of CMV-specific CD8+ T cells producing individual cytokines: IFNγ (red), TNF (green) and IL2 (blue). Error bars represent the mean±SEM *, P<0.05; **, P<0.01; ***, P<0.001, ns=not significant (determined by the student t test).

FIG. 12: Assessment of long-term durable CMV gB-specific CD4+ T cell immunity induced by CMV gB, CMVpoly20PL-NH and CpG 1018 vaccine formulation in HLA A24 transgenic mice. Human HLA A24 transgenic mice were immunized with CMV gB (5 μg) and CMVpoly20PL-NH (30 μg) formulated with CpG 1018 (50 μg) on day 0. On day 21 mice were tail bled and boosted with an identical vaccine formulation on day 28 and 42 and 210. Mice were sacrificed on day 28, 42, 49, 84, 133, 203 and 217 to perform longitudinal analysis to assess CMV gB-specific CD4+ T cells immune responses. On the day of sacrifice splenocytes were prepared and then stimulated with CMV gB pepmix consisting of a pool of 224 peptides (15mers with 11 aa overlap) in the presence of brefeldin A and the CMV-specific CD8+ T cells producing IFN-γ was measured using an ICS assay. Evaluation of CMV gB-specific CD4+ T cell responses following in vitro stimulation. Following immunization splenocytes were in vitro stimulated with CMV gB pepmix consisting of a pool of 224 peptides (15mers with 11 aa overlap) and cells were cultured for 10 days. On day 10, T cell specificity was assessed using ICS assay. (A and B) shows percentage of CMV gB-specific CD4+ T cells producing IFN-γ following ex vivo and in vitro stimulation. (C) shows SPICE analysis of CMV-specific CD4+ T cells producing proportion of the responses comprised of various combinations of IFNγ⁺/TNF⁺/IL2⁺ cytokines. Arc indicates the proportion of CMV-specific CD4+ T cells producing individual cytokines: IFNγ (red), TNF (green) and IL2 (blue). Error bars represent the mean±SEM *, P<0.05; **, P<0.01; ***, P<0.001, ns=not significant (determined by the student t test).

FIG. 13: Assessment of long-term durable germinal center B cell responses induced by CMV gB, CMVpoly20PL-NH and CpG 1018 vaccine formulation in HLA A24 transgenic mice: Human HLA A24 transgenic mice were immunized with CMV gB (5 μg) and CMVpoly20PL-NH (30 μg) formulated with CpG 1018 (50 μg) on day 0. On day 21 mice were tail bled and boosted with an identical vaccine formulation on day 28 and 42 and 210. Mice were sacrificed on day 28, 42, 49, 84, 133, 203 and 217 to perform longitudinal analysis to assess germinal center B cell responses immune responses in human HLA A24 transgenic mice. On the day of sacrifice single cell suspensions were prepared from spleens and lymph nodes and then stained with PE conjugated anti-B220, FITC conjugated anti-GL7 and APC conjugated anti-CD95 antibodies. Cells were analysed on the FACS to determine the frequencies of germinal center B cells. (A) shows frequencies of germinal centre B cells in human HLA A24 transgenic mice vaccinated with CMV vaccine or CpG 1018 alone on Day 28, 42, 49, 84, 133, 203 and 217 in spleens. (B) shows representative FACS plots from one vaccinated and control mice on day 28 and 49. (C) shows frequencies of germinal centre B cells in human HLA A24 transgenic mice vaccinated with CMV vaccine or CpG 1018 alone on day 28, 42, 49, 84, 133, 203 and 217 in lymph nodes. (D) shows representative FACS plots from one vaccinated and control mice on day 28 and 49.

FIG. 14: Assessment of long-term durable CMV gB-specific antibody secreting B cell responses induced by CMV gB, CMVpoly20PL-NH and CpG 1018 vaccine formulation in HLA A24 transgenic mice: Human HLA A24 transgenic mice were immunized with CMV gB (5 μg) and CMVpoly20PL-NH (30 μg) formulated with CpG 1018 (50 μg) on day 0. On day 21 mice were tail bled and boosted with an identical vaccine formulation on day 28 and 42 and 210. Mice were sacrificed on day 28, 42, 49, 84, 133, 203 and 217 to perform longitudinal analysis to assess CMV gB-specific antibody secreting B cell immune responses. On the day of sacrifice splenocytes were prepared and then assessed their ability to secrete gB-specific antibodies using ELISpot assay. A and B shows number of antibody secreting B cells/3×105 splenocytes in mice immunised with CMV vaccine formulation (V) or CpG 1018 (C) control on day 28, 42, 49, 84, 133, 203 and 217 following ex vivo and memory B cell ELISpot analysis. Error bars represent the mean±SEM *, P<0.05; **, P<0.01; ***, P<0.001, ****, P<0.0001 ns=not significant (determined by the student t test).

FIG. 15: Assessment of long-term durable CMV gB-specific antibody responses induced by CMV gB, CMVpoly20PL-NH and CpG 1018 vaccine formulation in HLA A24 transgenic mice. Human HLA A24 transgenic mice were immunized with CMV gB (5 μg) and CMVpoly20PL-NH (30 μg) formulated with CpG 1018 (50 μg) on day 0. On day 21 mice were tail bled and boosted with an identical vaccine formulation on day 28 and 42 and 210. Mice were sacrificed on day 28, 42, 49, 84, 133, 203 and 217 to perform longitudinal analysis to assess CMV gB-specific antibody responses using ELISA. On the day of sacrifice serum samples were collected and analysed using ELISA to determine the gB-specific antibody titres. (A) shows line graph of CMV gB-specific antibody titres in serum samples of human HLA A24 transgenic mice immunised with CMV vaccine (V) or CpG 1018 alone (C) on day 21, 28, 42, 49, 84, 133, 203 and 217. (B) shows prevalence of CMV-gB specific immunoglobulin isotypes (in pooled serum samples) (IgA, IgM, IgG1, IgG2a, IgG2b and IgG3) induced following immunisation with CMV vaccine formulation or placebo on day 21, 28, 42, 49, 84, 133, 203 and 217. (C and D) shows 50% neutralising antibody titres induced following immunisation with CMV vaccine formulation or placebo on day 21, 28, 42, 49, 84, 133, 203 and 217. Neutralising antibody responses were measured by IE-1 staining against CMV AD 169 strain in fibroblasts and CMV TB40/E strain in ARPE 19 cells. (E and F) shows flow cytometry analysis of frequency of gB-specific antibodies induced following immunisation with CMV vaccine or placebo binding to fibroblasts infected with CMV AD169 strain.

FIG. 16: Confirmation of long-term durable CMV specific-immunity induced by CMV gB, CMVpoly20PL-NH and CpG 1018 vaccine formulation in HLA A1 transgenic mice: Human HLA A1 transgenic mice were immunized with CMV gB (5 μg) and CMVpoly20PL-NH (30 μg) formulated with CpG 1018 (50 μg) on day 0. On day 21 mice were tail bled and boosted with an identical vaccine formulation on day 28 and 42 and 210. Mice were sacrificed on day 49 and 84 to perform longitudinal analysis to assess CMV-specific T cell and humoral immune responses.

FIG. 17: Assessment of long-term durable CMV specific-CD8+ T cell immunity induced by CMV gB, CMVpoly20PL-NH and CpG 1018 vaccine formulation in HLA A1 transgenic mice. Human HLA A1 transgenic mice were immunized with CMV gB (5 μg) and CMVpoly20PL-NH (30 μg) formulated with CpG 1018 (50 μg) on day 0. On day 21 mice were tail bled and boosted with an identical vaccine formulation on day 28 and 42 and 210. Mice were sacrificed on day 49, 84, 203 and 217 to perform longitudinal analysis to assess CMV-specific CD8+ T cells immune responses. On the day of sacrifice single cell suspensions were prepared and then stimulated with respective CMV CD8+ T cell peptides (HLA A1-VTE & YSE) in the presence of brefeldin A and the CMV specific CD8+ T cells producing IFN-γ was measured using an ICS assay. Evaluating CMV-specific CD8+ T cell responses following in vitro stimulation. Following immunization splenocytes were in vitro stimulated with respective CMV CD8+ T cell 14 peptides (HLA A1-VTE & YSE) and cells were cultured for 10 days. On day 10, T cell specificity was assessed using ICS assay. (A and B) shows percentage of CMV-specific CD8+ T cells producing IFN-γ following ex vivo. (C) shows percentage of CMV-specific CD8+ T cells producing IFN-γ following in vitro stimulation with CMV CD8+ T cell peptides. (D) shows ability of CMV-specific CD8+ T cells to secrete multiple cytokines (CD107a⁺, IFNγ⁺, TNF or IL2⁺) measured by flow cytometry and intracellular cytokine staining following in vitro stimulation with CMV CD8+ T cell peptides. Pie chart represent the percentage of CMV-specific CD8+ T cells producing 4 cytokines (light blue), 3 cytokine (red), 2 cytokine (green) and 1 cytokine (purple) on day 49, 84, 203 and 217. Error bars represent the mean±SEM *, P<0.05. ns=not significant 5 (determined by the student t test).

FIG. 18: Assessment of long-term durable CMV gB-specific CD4+ T cell immunity induced by CMV gB, CMVpoly20PL-NH and CpG 1018 vaccine formulation in HLA A1 transgenic mice. Human HLA A1 transgenic mice were immunized with CMV gB (5 μg) and CMVpoly20PL-NH (30 μg) formulated with CpG 1018 (50 μg) on day 0. On day 21 mice were tail bled and boosted with identical vaccine formulation on day 28 and 42 and 210. Mice were sacrificed on day 49 and 84 to perform longitudinal analysis to assess CMV gB-specific CD4+ T cells immune responses. On the day of sacrifice splenocytes were prepared and then stimulated with CMV gB pepmix consisting of a pool of 224 peptides (15mers with 11 aa overlap) in the presence of brefeldin A and the CMV-specific CD4+ T cells producing IFN-γ was measured using an ICS assay. Evaluating CMV gB-specific CD4+ T cell responses in in vitro stimulated splenocytes. Following immunization splenocytes were in vitro stimulated with CMV gB pepmix consisting of a pool of 224 peptides (15mers with 11 aa overlap) and cells were cultured for 10 days. On day 10, T cell specificity was assessed using ICS assay. (A and B) shows percentage of CMV gB-specific CD4+ T cells producing IFN-γ following ex vivo. (C) shows percentage of CMV-specific CD4+ T cells producing IFN-γ following in vitro stimulation with CMV gB pepmix. (D) shows ability of CMV-specific CD4+ T cells secreting multiple cytokines (CD107a⁺, IFNγ⁺, TNF or IL2⁺) measured by flow cytometry and intracellular cytokine staining following in vitro stimulation of splenocytes with CMV gB pepmix. Pie charts represent the percentage of CMV-specific CD4+ T cells producing 4 cytokines (light blue), 3 cytokine (red), 2 cytokine (green) and 1 cytokine (purple) on day 49, 84, 203 and 217. Error bars represent the mean±SEM *, P<0.05; **, P<0.01, ns=not significant (determined by the student t test).

FIG. 19: Assessment of long-term durable germinal centre and antibody secreting B cell responses induced by CMV gB, CMVpoly20PL-NH and CpG 1018 vaccine formulation in HLA A1 transgenic mice: Human HLA A1 transgenic mice were immunised with CMV gB (5 μg) and CMVpoly20PL-NH (30 μg) formulated with CpG 1018 (50 μg) on day 0. On day 21 mice were tail bled and boosted with an identical vaccine formulation on day 28 and 42 and 210. Mice were sacrificed on day 49 and 84, to perform longitudinal analysis to assess vaccine induced germinal centre B cells and CMV gB30 specific antibody secreting B cell responses. On the day of sacrifice single cell suspensions were prepared from spleens and then stained with PE conjugated anti-B220, FITC conjugated anti-GL7 and APC conjugated anti-CD95 antibodies. Cells were analysed on the FACS to determine the frequencies of germinal centre B cells. (A and B) shows frequencies of germinal centre B cells in human HLA A1 transgenic mice vaccinated with CMV vaccine (V) or CpG 1018 alone (C) on day 49, 84, 204 and 217 in spleens. (B) shows representative FACS plots from one vaccinated and control mice on day 49 and 84. (C and D) shows number of antibody secreting B cells/3×10⁵ splenocytes from immunised with CMV vaccine formulation (V) or CpG 1018 (C) control mice on day 49, 84 203 and 217 following ex vivo B cell ELISpot analysis. To determine the antibody secreting memory B cells, splenocytes were in vitro stimulated with recombinant mouse IL2 and R848 for 4 days and then measured their ability to secrete CMV gB-specific antibodies. (E and F) shows number of antibody secreting B cells/3×10⁵ splenocytes from immunised with CMV vaccine formulation (V) or CpG 1018 (C) control mice on day 49, 84 203 and 217 following memory B cell ELISpot analysis. Error bars represent the mean±SEM *, P<0.05; **, P<0.01; ***, P<0.001, ****, P<0.0001 ns=not significant (determined by the student t test).

FIG. 20: Assessment of long-term durable CMV gB-specific antibody responses induced by CMV gB, CMVpoly20PL-NH and CpG 1018 vaccine formulation in HLA A1 transgenic mice. Human HLA A1 transgenic mice were immunized with CMV gB (5 μg) and CMVpoly20PL-NH (30 μg) formulated with CpG 1018 (50 μg) on day 0. On day 21 and 42 mice were tail bled and boosted with an identical vaccine formulation on day 21 and 42 and 210. Mice were sacrificed on day 49, 84, 203 and 217 to perform longitudinal analysis to assess CMV gB-specific antibody responses using ELISA. On the day of sacrifice serum samples were collected and analysed using ELISA to determine the gB-specific antibody titres. (A) shows line graph of CMV gB-specific antibody titres in serum samples of human HLA A1 transgenic mice immunised with CMV vaccine (V) or CpG 1018 alone (C) on day 21, 28, 42 49, 84, 203 and 217. (B) shows prevalence of CMV-gB specific immunoglobulin (in pooled serum samples) isotypes (IgA, IgM, IgG1, IgG2a, IgG2b and IgG3) induced following immunisation with CMV vaccine formulation or placebo on day 21, 28, 42, 49, 84, 203 and 217.

### BRIEF DESCRIPTION OF THE SEQUENCES

SEQ ID NO: 1=amino acid sequence of pp65 epitope (HLA B35) in Table 1 (epitope sequence in black; FPTKDVAL).

SEQ ID NO: 2=amino acid sequence of pp65 epitope (HLA A11) in Table 1 (epitope sequence in black; GPISHGHVLK).

SEQ ID NO: 3=amino acid sequence of pp65 epitope (HLA A24) in Table 1 (epitope sequence in black; QYDPVAALF).

SEQ ID NO: 4=amino acid sequence of pp65 epitope (HLA A1) in Table 1 (epitope sequence in black; YSEHPTFTSQY).

SEQ ID NO: 5=amino acid sequence of pp65 epitope (HLA B7) in Table 1 (epitope sequence in black; TPRVTGGGAM).

SEQ ID NO: 6=amino acid sequence of IE-1 epitope (HLA B8) in Table 1 (epitope sequence in black; QIKVRVDMV).

SEQ ID NO: 7=amino acid sequence of pp65 epitope (HLA B35) in Table 1 (epitope sequence in black; IPSINVHHY).

SEQ ID NO: 8=amino acid sequence of pp150 epitope (HLA A3) in Table 1 (epitope sequence in black; TTVYPPSSTAK).

SEQ ID NO: 9=amino acid sequence of pp65 epitope (HLA B7) in Table 1 (epitope sequence in black; RPHERNGFTVL).

SEQ ID NO: 10=amino acid sequence of IE-1 epitope (HLA B8/B18/B44) in Table 1 (epitope sequence in black; DELRRKMMYM).

SEQ ID NO: 11=amino acid sequence of pp50 epitope (HLA A1) in Table 1 (epitope sequence in black; VTEHDTLLY).

SEQ ID NO: 12=amino acid sequence of pp65 epitope (HLA A2) in Table 1 (epitope sequence in black; NLVPMVATV).

SEQ ID NO: 13=amino acid sequence of IE-1 epitope (HLA A2) in Table 1 (epitope sequence in black; VLEETSVML).

SEQ ID NO: 14=amino acid sequence of pp65 epitope (HLA A23/A24) in Table 1 (epitope sequence in black; AYAQKIFKIL).

SEQ ID NO: 15=amino acid sequence of pp65 epitope (HLA CW6) in Table 1 (epitope sequence in black; TRATKMQVI).

SEQ ID NO: 16=amino acid sequence of DNAse epitope (HLA B27) in Table 1 (epitope sequence in black; ARVYEIKCR).

SEQ ID NO: 17=amino acid sequence of IE-1 epitope (HLA B40) in Table 1 (epitope sequence in black; KEVNSQLSL).

SEQ ID NO: 18=amino acid sequence of IE-1 epitope (HLA B8) in Table 1 (epitope sequence in black; ELKRKMIYM).

SEQ ID NO: 19=amino acid sequence of IE-1 epitope (HLA A2) in Table 1 (epitope sequence in black; YILEETSVML).

SEQ ID NO: 20=amino acid sequence of pp65 epitope (HLA B57/B58) in Table 1 (epitope sequence in black; QAIRETVEL).

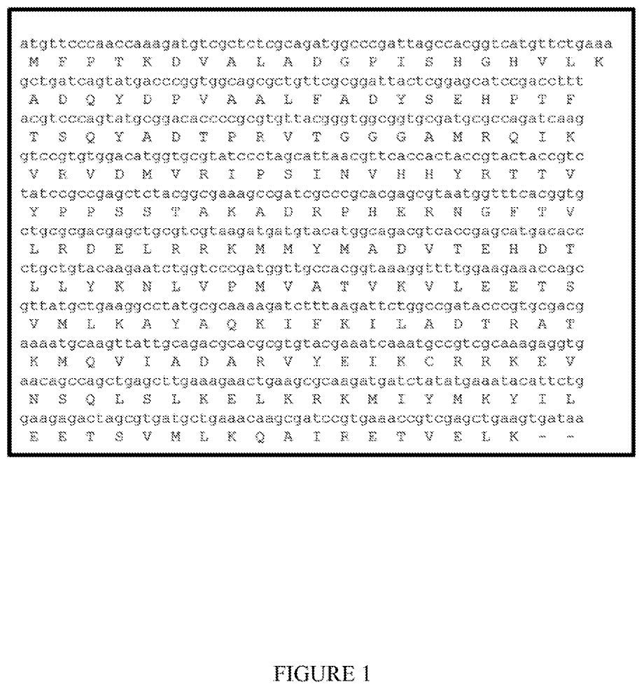

SEQ ID NO: 21=amino acid sequence of CMVpoly20PL-NH polytope protein in FIG. 1.

SEQ ID NO: 22=nucleotide sequence of CMVpoly20PL-NH polytope protein in FIG. 1.

### DETAILED DESCRIPTION OF THE INVENTION

The present invention is at least partly predicated on the unexpected discovery that a protein-based vaccine including an isolated protein that comprises a plurality of CMV epitopes, a CMV envelope protein and a TLR9 agonist when administered to an individual may elicit a protective and durable immune response therein. In this regard, the vaccine formulation of the invention advantageously induces the generation of long-lived germinal centre B cells, long-term sustainable CMV-specific CD4+ and CD8+ T cells, an antibody secreting B cell response and neutralising antibody responses.

In one aspect, the invention relates to a pharmaceutical composition comprising:

(a) one or a plurality of isolated proteins comprising a plurality of epitopes, wherein the plurality of epitopes are derived from two or more different CMV antigens;

(b) a CMV envelope protein, or a fragment, variant or derivative thereof; and

(c) a TLR agonist, such as a TLR9 agonist.

By “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state.

In the context of the present invention, an “exogenous” protein or polyepitope protein is a protein produced externally to the animal to which it is subsequently administered. Effectively, the exogenous protein is administered or administrable to the animal, rather than being produced or expressed by the animal in situ (e.g. by cells or tissues of the animal) following delivery of a nucleic acid or genetic construct encoding the protein to the animal. A preferred exogenous protein is a recombinant protein produced in an isolated host cell ex vivo, such as a bacterial host cell.

By “protein” is meant an amino acid polymer comprising natural and/or non-natural amino acids, D- or L-amino acids as are well known in the art.

A “peptide” is a protein having no more than fifty (50) amino acids.

A “polypeptide” is a protein having more than fifty (50) amino acids.

The isolated proteins described herein, inclusive of fragments, variants and derivatives thereof, may be produced by any means known in the art, including but not limited to, chemical synthesis, recombinant DNA technology and proteolytic cleavage to produce peptide fragments.

Chemical synthesis is inclusive of solid phase and solution phase synthesis. Such methods are well known in the art, although reference is made to examples of chemical synthesis techniques as provided in Chapter 9 of SYNTHETIC VACCINES Ed. Nicholson (Blackwell Scientific Publications) and Chapter 15 of CURRENT PROTOCOLS IN PROTEIN SCIENCE Eds. Coligan et al., (John Wiley & Sons, Inc. NY USA 1995-2008). In this regard, reference is also made to International Publication WO 99/02550 and International Publication WO 97/45444.

Recombinant proteins may be conveniently prepared by a person skilled in the art using standard protocols as for example described in Sambrook et al., MOLECULAR CLONING. A Laboratory Manual (Cold Spring Harbor Press, 1989), in particular Sections 16 and 17; CURRENT PROTOCOLS IN MOLECULAR BIOLOGY Eds. Ausubel et al., (John Wiley & Sons, Inc. NY USA 1995-2008), in particular Chapters 10 and 16; and CURRENT PROTOCOLS IN PROTEIN SCIENCE Eds. Coligan et al., (John Wiley & Sons, Inc. NY USA 1995-2008), in particular Chapters 1, 5 and 6. Typically, recombinant protein preparation includes expression of a nucleic acid encoding the protein in a suitable host cell.

As used herein, the term “epitope” refers to any protein determinant capable of specific binding to an immunoglobulin or fragment thereof, or a T-cell receptor.

In particular embodiments, the epitope is or comprises a cytotoxic T-lymphocyte (CTL) epitope. A “CTL epitope” is a peptide, or an amino acid sequence of the peptide, that is capable of stimulating or activating a cytotoxic T lymphocyte to recognize a target cell presenting the epitope in the context of the appropriate MHC Class I molecule. Recognition of the target cell may include or result in cytokine production (e.g., IFN-γ, IL-2, MIP-1β and/or TNF), changes in cell surface marker expression (e.g. CD107a) and/or lysis and/or killing of the target cell. Given this, the composition described herein may be capable of inducing or eliciting a cytotoxic T-lymphocyte immune response upon administration to a subject.

In another embodiment, the isolated proteins, including a polyepitope protein, may further comprise one or a plurality of CD4⁺ helper T cell epitopes and/or B cell epitopes.

In further embodiments, the isolated proteins, including the polyepitope protein, may further comprise one or a plurality of HLA Class II restricted CTL epitopes.

Furthermore, it will be appreciated that the particular number and/or type of the constituent epitopes of the pharmaceutical composition may readily be altered while retaining broad HLA Class I-restricted immunogenicity.

Typically, although not exclusively, an epitope, such as a CTL epitope, comprises 7, 8, 9, 10, 11, 12, 13, 14 or 15 contiguous amino acids of, derived from, obtained from or based on a corresponding CMV antigen.

It will be appreciated by a skilled person that the epitope selected for inclusion in the pharmaceutical composition may be tailored to fit any population, race or other group of individuals.

Other criteria for inclusion of particular epitopes within the pharmaceutical composition include those (i) having minimal or no sequence variants; (ii) selected from HLAs having minimal subtypes; and (iii) having a high frequency of CTL responses in healthy seropositives.

The isolated proteins, including the polytope protein described herein, preferably comprise a plurality of CMV epitopes derived from a plurality of different CMV protein antigens. Preferably, the epitopes are of CMV antigens selected from the group consisting of: pp50, pp65, pp150, DNAse, IE-1 and any combination thereof.

Suitably, the one or plurality of isolated proteins, including the polytope protein, comprise epitopes, such as CTL epitopes, selected to provide broad coverage of a population. In humans, these include HLA class I specificities HLA-A1, -A2, -A3, -A11, -A23, -A24, -A26, -A29, -A30, -B7, -B8, -B18, -B27, -B35, -B38, -B40, -B41, -B44, -B51, -B57, -B58 and -CW6. In certain embodiments, the epitopes are restricted to the HLA class I specificities shown in Table 1. Accordingly, in one embodiment, the epitopes are restricted by one or a plurality of HLA class I specificities selected from HLA-A1, -A2, -A3, -A11, -A23, -A24, -B7, -B8, -B18, -B27, -B35, -B40, -B44, -B57, -B58 and -CW6.

In a particular embodiment, the isolated proteins, inclusive of the polytope protein, comprises a plurality of HLA class I restricted epitopes selected from Table 1 (SEQ ID NOS: 1-20). In one particular embodiment, the one or plurality of isolated proteins or polytope protein comprise each of the epitope amino acid sequences set forth in SEQ ID NOS: 1-20 or a fragment, variant or derivative thereof.

In some embodiments, said plurality of epitopes comprises thirty (30) or less (e.g., 30, 29, 28, 27, 26, 25, 24, 23, 22, 21, 20, 19, 18, 17, 16, 15, 14, 13, 12, 11, 10 etc. or any range therein) epitopes in total. In other embodiments, said plurality of epitopes comprises twenty (20) or less (e.g., 20, 19, 18, 17, 16, 15, 14, 13, 12, 11, 10 etc. or any range therein) epitopes in total.

It will also be appreciated that other CMV epitopes in addition to those provided in Table 1 may be used, such as those described in International Publication WO 03/000720 and International Publication No. WO2014/059489.

In particular embodiments, the one or plurality of isolated proteins of the pharmaceutical composition include a plurality of separate isolated proteins, each of which comprise only one of the plurality of epitopes. It will also be appreciated, however, that the invention contemplates the inclusion of one or more isolated proteins that include two or more of the plurality of epitopes derived from the same or different CMV antigen with or without the further inclusion of one or more isolated proteins that include only one of the plurality of epitopes.

Accordingly, the one or plurality of isolated proteins may be or comprise an isolated polyepitope or polytope protein. For example, an isolated “CMV polyepitope” or an isolated “CMV polyepitope protein”. In this regard, the polytope protein suitably comprises two or more of the plurality of epitopes from the two or more different CMV antigens, as hereinafter described.

In addition to the epitopes, the polytope protein may further comprise intervening amino acids or amino acid sequences. Intervening amino acids or amino acid sequences may be present between at least two of the epitope amino acid sequences, or between each adjacent epitope amino acid sequence.

Suitably, the intervening amino acids or amino acid sequences are positioned or located relative to the epitope amino acid sequences to enable proteasomal processing and for transporting the proteasome-generated, individual epitope peptides into the endoplasmic reticulum (ER) for subsequent presentation with HLA-I molecules.

In one embodiment, the intervening amino acids or amino acid sequences are proteasome liberation amino acids or amino acid sequences. Non-limiting examples of proteasome liberation amino acids or amino acid sequences are or comprise AD, K or R.

In one form, the CMV epitopes are linked or joined by the proteasome liberation amino acid sequence at the carboxyl terminus of each respective epitope of the polytope protein.

Once administered, the polytope protein comprising the intervening amino acids or amino acid sequences is processed, such as by a proteasomal pathway. This results in HLA Class I-dependent presentation of the processed CMV epitopes to CD8⁺ cytotoxic T cells.

For the polytope protein, the inclusion of particular epitopes can be based on epitope hydrophobic properties, wherein the sequential order of individual epitopes is arranged such that hydrophobicity is uniformly distributed along the length of the polyepitope to assist inter cellular mobility.

An embodiment of a full length, contiguous polyepitope protein that may be included in the pharmaceutical composition of the present aspect is set forth in SEQ ID NO:21. It will be further appreciated that other polytope proteins that comprises CMV epitopes may be included in the pharmaceutical composition of the present aspect, such as those described in International Publication No. WO2014/059489, which is incorporated by reference in its entirety herein.

Suitably, the pharmaceutical composition of the present aspect also includes one or more CMV envelope proteins. As will be appreciated, viral envelope proteins typically participate in virus binding to and/or entry of the infectious virus into a target cell. The term “CMV envelope protein” refers to the protein(s) embedded in the membrane which surrounds the CMV viral nucleocapsid.

In particular embodiments, the CMV envelope protein is a CMV envelope glycoprotein. CMV envelope glycoproteins represent attractive vaccine candidates as they are expressed on the viral surface and can elicit protective virus-neutralizing humoral immune responses. Exemplary, CMV envelope glycoproteins include glycoprotein B (gB), glycoprotein H (gH), glycoprotein L (gL), glycoprotein M (gM), glycoprotein N (gN) and glycoprotein O (gO). In one particular embodiment, the CMV envelope protein is or comprises glycoprotein B.

Fragments, variants and derivatives of gB, such as those hereinbefore described and as are known in the art, are also envisaged as suitable for inclusion in the pharmaceutical composition of the present aspect. By way of example, CMV glycoprotein B protein may include a modified extracellular domain or a modified endoproteolytic cleavage site. Additionally, CMV gB may be truncated, such as C-terminally truncated including deletion of a transmembrane and/or cytoplasmic domain thereof. CMV gB variants can include naturally occurring (e.g., allelic, strain-specific) variants, orthologs (e.g., from a different herpes virus) and synthetic variants, such as produced in vitro using mutagenesis techniques. Suitably, such fragments, variants and derivatives of gB are capable of eliciting an immune response, such as a cell mediated and/or humoral immune response, when administered to a subject. In this regard, fragments, variants and derivatives of gB suitable include one or more T-cell epitopes and/or B-cell epitopes.

Based on the above, it will also be appreciated that the one or plurality of isolated proteins, including the polytope protein, and/or the CMV envelope protein described herein may be subjected to further modifications, variations and/or derivatizations without departing from the inventive concept.

Variations in amino acid sequence may be the result of naturally occurring sequence variation in a CMV epitope or a CMV envelope protein.

It is well understood in the art that some amino acids may be changed to others with broadly similar properties without changing the nature of the activity of the isolated protein (conservative substitutions). Typically, conservative substitutions are made so that amino acid properties such as charge, hydrophilicity, hydrophobicity and/or side chain size or “bulkiness” are retained or at least minimally altered.

Introduction of amino acid substitutions may be readily achieved during peptide synthesis or by mutagenesis of an encoding nucleic acid. Non-limiting examples of nucleic acid mutagenesis methods are provided in Chapter 9 of CURRENT PROTOCOLS IN MOLECULAR BIOLOGY, Ausubel et al., supra, Stemmer, 1994, Proc. Natl. Acad. Sci. USA 91 10747, Shafikhani et al., 1997, Biotechniques 23 304, Jenkins et al., 1995, EMBO J. 14 4276-4287 and Zaccolo et al., 1996, J. Mol. Biol. 255 58 and kits such as QuickChange™ Site-Directed Mutagenesis Kit (Stratagene) and the Diversify™ random mutagenesis kit (Clontech).

Generally, the invention contemplates protein variants having at least 75%, preferably at least 80%, more preferably at least 85% or even more preferably at least 90, 91, 92, 93, 94, 95, 96, 97, 98 or 99% amino acid sequence identity with the constituent epitope sequences, individually or in combination or the CMV envelope protein. In other embodiments, this may include conservative variations or substitutions of one (1), two (2) or three (3) amino acid residues of a CMV epitope or a CMV envelope protein.

The term “sequence identity” is used herein in its broadest sense to include the number of exact amino acid matches having regard to an appropriate alignment using a standard algorithm, having regard to the extent that sequences are identical over a window of comparison. Sequence identity may be determined using computer algorithms such as GAP, BESTFIT, FASTA and the BLAST family of programs as for example disclosed by Altschul et al., 1997, Nucl. Acids Res. 25 3389. A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

As used herein, “derivatives” are molecules such as proteins, fragments or variants thereof that have been altered, for example by conjugation or complexing with other chemical moieties, by post-translational modification (e.g. phosphorylation, acetylation and the like), modification of glycosylation (e.g. adding, removing or altering glycosylation), lipidation and/or inclusion of additional amino acid sequences as would be understood in the art.

Additional amino acid sequences may include fusion partner amino acid sequences which create a fusion protein. By way of example, fusion partner amino acid sequences may assist in detection and/or purification of the isolated fusion protein. Non-limiting examples include metal-binding (e.g. polyhistidine) fusion partners, maltose binding protein (MBP), Protein A, glutathione S-transferase (GST), fluorescent protein sequences (e.g. GFP), epitope tags such as myc, FLAG and haemagglutinin tags.

Other additional amino acid sequences may be of carrier proteins such as diphtheria toxoid (DT) or a fragment thereof, or a CRM protein fragment such as described in International Publication WO2017/070735.

Other derivatives contemplated by the invention include, but are not limited to, modification to side chains, incorporation of unnatural amino acids and/or their derivatives during peptide, or protein synthesis and the use of crosslinkers and other methods which impose conformational constraints on the isolated proteins of the invention.

In this regard, the skilled person is referred to Chapter 15 of CURRENT PROTOCOLS IN PROTEIN SCIENCE, Eds. Coligan et al. (John Wiley & Sons NY 1995-2008) for more extensive methodology relating to chemical modification of proteins.

The pharmaceutical composition of the present aspect suitably includes one or more TLR agonists for administration to the subject.

The term “TLR agonist”, as used herein, refers to a molecule that is capable of causing a signalling response through a TLR signalling pathway, either as a ligand directly or indirectly through of the generation of endogenous or exogenous ligand. The agonist ligands of the TLR receptors can be natural ligands of the TLR receptor, or functionally equivalent variants thereof that retain the ability to bind to the TLR receptor and induce costimulation signals therein. The TLR agonist may also be an agonist antibody against the TLR receptor, or a functionally equivalent variant thereof, that is capable of specifically binding to the TLR receptor and, more particularly, to the extracellular domain of said receptor, and inducing some of the immune signals controlled by this receptor and associated proteins. The specificity of binding can be for the human TLR receptor or for a human homologous TLR receptor of a different species.

In particular embodiments, the one or more TLR agonists include a TLR4 agonist and/or a TLR9 agonist. More particularly, the TLR agonist is or comprises a TLR9 agonist.

Exemplary TLR4 agonists are lipolopysacchardides (LPS) or derivatives or components of LPS. These include Monophosphoryl lipid A (MPL®) derived from Salmonella minnesota and synthetic TLR4 agonists such as aminoalkyl glucosaminide phosphates (AGPs) and Phosphorylated HexaAcyl Disaccharide (PHAD) and derivatives thereof (e.g., 3D-PHAD, 3D(6-acyl)-PHAD). A preferred TLR4 agonist is MPL.

TLR9 recognizes specific unmethylated CpG oligonucleotides (ODN) sequences that distinguish microbial DNA from mammalian DNA. CpG ODNs oligonucleotides contain unmethylated CpG dinucleotides in particular sequence contexts (CpG motifs). These CpG motifs are present at a 20-fold greater frequency in bacterial DNA compared to mammalian DNA. Three types of stimulatory ODNs have been described: type A, B and C.

Non-limiting examples of TLR9 agonists include CpG ODN1018, CpG ODN2006, CpG ODN2216, CpG ODN1826 and CpG ODN2336, although without limitation thereto. In some embodiments, the TLR9 agonist is or comprises CpG ODN1018 and/or CpG ODN2006. In one preferred embodiment, the TLR9 agonist is CpG ODN1018.

In particular embodiments, the TLR agonist is not MPL, CpG ODN1826, CpG ODN2006, CpG ODN2216 and/or CpG ODN2336.

Suitably, the pharmaceutical composition further comprises a pharmaceutically-acceptable carrier, diluent or excipient.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, organic acids such as acetates, propionates and malonates and pyrogen-free water.

A useful reference describing pharmaceutically acceptable carriers, diluents and excipients is Remington's Pharmaceutical Sciences (Mack Publishing Co. N.J. USA, 1991) which is incorporated herein by reference.

It will be appreciated by the foregoing that the pharmaceutical composition of the invention may include an “immunologically-acceptable carrier, diluent or excipient”.

Useful carriers are well known in the art and include for example: thyroglobulin; albumins such as human serum albumin; toxins, toxoids or any mutant crossreactive material (CRM) of the toxin from tetanus, diphtheria, pertussis, Pseudomonas, E. coli, Staphylococcus, and Streptococcus; polyamino acids such as poly(lysine:glutamic acid); influenza; Rotavirus VP6, Parvovirus VP1 and VP2; hepatitis B virus core protein; hepatitis B virus recombinant vaccine and the like. Alternatively, a fragment or epitope of a carrier protein or other immunogenic protein may be used. For example, a T cell epitope of a bacterial toxin, toxoid or CRM may be used. In this regard, reference may be made to U.S. Pat. No. 5,785,973 which is incorporated herein by reference.

The “immunologically-acceptable carrier, diluent or excipient” includes within its scope water, bicarbonate buffer, phosphate buffered saline or saline and/or an adjuvant as is well known in the art. As will be understood in the art, an “adjuvant” means a composition comprised of one or more substances that enhances the immunogenicity and efficacy of a vaccine composition.

Accordingly, in addition to the TLR agonist, the pharmaceutical compositions, immunogenic compositions and/or vaccines described herein may include one or more further immunostimulatory molecules or adjuvants for administration to a subject.

Suitable immunostimulatory molecules and adjuvants include, but are not limited to: a further TLR agonist, lipopolysaccharide and derivatives thereof such as MPL, Freund's complete or incomplete adjuvant, squalane and squalene (or other oils of plant or animal origin); block copolymers; detergents such as Tween®-80; Quil® A, mineral oils such as Drakeol or Marcol, vegetable oils such as peanut oil; Corynebacterium-derived adjuvants such as Corynebacterium parvum; Propionibacterium-derived adjuvants such as Propionibacterium acne; Mycobacterium bovis (Bacille Calmette and Guerin or BCG); Bordetella pertussis antigens; tetanus toxoid; diphtheria toxoid; surface active substances such as hexadecylamine, octadecylamine, octadecyl amino acid esters, lysolecithin, dimethyldioctadecylammonium bromide, N,N-dicoctadecyl-N′,N′bis(2-hydroxyethyl-propanediamine), methoxyhexadecylglycerol, and pluronic polyols; polyamines such as pyran, dextransulfate, poly IC carbopol; peptides such as muramyl dipeptide and derivatives, dimethylglycine, tuftsin; oil emulsions; and mineral gels such as aluminium phosphate, aluminium hydroxide or alum; interleukins such as interleukin 2 and interleukin 12; monokines such as interleukin 1; tumour necrosis factor; interferons such as gamma interferon; combinations such as saponin-aluminium hydroxide or Quil-A aluminium hydroxide; liposomes; ISCOM® and ISCOMATRIX® adjuvant; mycobacterial cell wall extract; synthetic glycopeptides such as muramyl dipeptides or other derivatives; Avridine; Lipid A derivatives; dextran sulfate; DEAE-Dextran alone or with aluminium phosphate; carboxypolymethylene such as Carbopol′ EMA; acrylic copolymer emulsions such as Neocryl A640 (e.g. U.S. Pat. No. 5,047,238); water in oil emulsifiers such as Montanide ISA 720; poliovirus, vaccinia or animal poxvirus proteins; or mixtures thereof.

With regard to subunit vaccines, an example of such a vaccine may be formulated with ISCOMs, such as described in International Publication WO97/45444.

An example of a vaccine in the form of a water-in-oil formulation includes Montanide ISA 720, such as described in International Publication WO97/45444.

Any suitable procedure is contemplated for producing vaccine compositions. Exemplary procedures include, for example, those described in New Generation Vaccines (1997, Levine et al., Marcel Dekker, Inc. New York, Basel, Hong Kong), which is incorporated herein by reference.

Any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular and transdermal administration may be employed.

Dosage forms include tablets, dispersions, suspensions, injections, solutions, syrups, troches, capsules, suppositories, aerosols, transdermal patches and the like. These dosage forms may also include injecting or implanting controlled releasing devices designed specifically for this purpose or other forms of implants modified to act additionally in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Preferred pharmaceutical compositions are “immunogenic compositions” that elicit a humoral and/or cell mediated immune response to thereby provide prophylactic and/or therapeutic treatment of a CMV infection or a CMV-associated disease, disorder or condition responsive to such immunotherapy, without necessarily eliciting a protective immune response.

In a preferred form, the immunogenic composition may be a vaccine for eliciting a protective B-lymphocyte- and/or CD8⁺ CTL-based immune response in a human subject that protects against a CMV infection, or treats an existing CMV infection or CMV-associated disease, disorder or condition. In particular embodiments, the immunogenic composition elicits a durable immune response in a subject that protects against CMV infection for greater than about 6 months (e.g., 6, 7, 8, 9, 10, 11, 12, 13, 14, 15, 16, 17, 18, 19, 20, 21, 22, 23, 24 etc. months or any range therein) following administration thereof.

As generally used herein the terms “immunize”, “vaccinate” and “vaccine” refer to methods and/or compositions that elicit a protective immune response against CMV, whereby subsequent infection by CMV is at least partly prevented or minimized.

Accordingly, such compositions may be delivered for the purposes of generating at least partial immunity, and preferably protective immunity, or for generating an immune response, preferably a protective immune response, to a CMV infection, whether new, existing or latent, upon administration to a subject, although without limitation thereto.

By “protective immunity” is meant a level of immunity whereby the responsiveness to an antigen or antigens is sufficient to lead to rapid binding and/or elimination of said antigens and thus at least partially ameliorate or prevent a subsequent CMV infection in an animal, such as human subjects.

By “protective immune response” is meant a level of immune response that is sufficient to prevent or reduce the severity, symptom, aspect, or characteristic of a current and/or future CMV infection in an animal, such as human subjects.

The term “immune response” refers to any response to an antigen or antigenic determinant by the immune system of a subject (e.g., a human). Exemplary immune responses include humoral immune responses (e.g., production of antigen-specific antibodies) and cell-mediated immune responses (e.g., production of antigen-specific T cells). Assays for assessing an immune response are known in the art and may comprise in vivo assays, such as assays to measure antibody responses and delayed type hypersensitivity responses. By way of example, the assay to measure antibody responses may measure B-cell function as well as B-cell/T-cell interactions. For the antibody response assay, antibody titres in the blood may be compared following an antigenic challenge. The in vitro assays may comprise determining the ability of immune cells to divide, or to provide help for other cells to divide, or to release lymphokines and other factors, express markers of activation, and lyse target cells. Immune cells, such as T lymphocytes, may also be tested in vitro for their ability to proliferate using mitogens or specific antigens. Supernatant from cultured immune cells can also be tested to quantitate the ability of the immune cells to secrete specific lymphokines. Moreover, the immune cells can be removed from culture and tested for their ability to express activation antigens. This can be done by any method that is suitable as in the non-limiting example of using antibodies or ligands which bind to the activation antigen as well as probes that bind the RNA coding for the activation antigen.

The term “humoral immune response” refers to the stimulation of antibody, including neutralizing antibody, production. Humoral immune response also refers to the accessory proteins and events that accompany antibody production, including T helper cell activation and cytokine production, affinity maturation, and memory cell generation.

The term “cell-mediated immune response” refers to the immunological defence provided by lymphocytes, such as the defence provided by sensitized T lymphocytes when they directly lyse cells expressing foreign antigens and secrete cytokines (e.g., IFN-gamma), which can modulate macrophage and natural killer (NK) cell effector functions and augment T cell expansion and differentiation.

As will be described in more detail in the Examples, the pharmaceutical composition of the present aspect is highly efficient in generating CMV-specific CD4⁺ and CD8+ T cell responses, B cell responses and neutralising antibody responses in multiple human HLA transgenic mice. Furthermore, the pharmaceutical composition is capable of inducing the generation of long-lived CMV-specific germinal centre B cells. It is proposed that these functional characteristics of the immune response produced by the pharmaceutical composition of the invention are important for the efficacy and durability of the resultant humoral and cell-mediated immune responses and hence subsequent virus clearance.

Generally, pharmaceutical compositions, immunogenic compositions, vaccines and/or methods of prophylactic or therapeutic treatment described herein may employ any safe route of administration may be employed for providing a patient with the composition of the invention. For example, oral, rectal, parenteral, sublingual, buccal, intravenous, intra-articular, intra-muscular, intra-dermal, subcutaneous, inhalational, intraocular, intraperitoneal, intracerebroventricular, transdermal and the like may be employed. Intra-muscular and subcutaneous injection is appropriate, for example, for administration of immunogenic compositions, proteinaceous vaccines and DNA vaccines.

It will be appreciated that the pharmaceutical composition of the present aspect may be useful in therapeutic and/or prophylactic treatment of a CMV-associated disease, disorder or condition in animals, preferably humans. In particular embodiments, the pharmaceutical composition described herein is for use in: (a) treating or preventing a CMV infection in a subject; (b) eliciting an immune response to a CMV antigen in a subject; and/or (c) inducing immunity against a CMV infection in a subject.

In humans, CMV infection can cause a mononucleosis-like syndrome with prolonged fever, and/or a mild hepatitis. In certain high-risk groups, disease can be more severe, such as during infection of the unborn baby during pregnancy, in people who work with children, and in immunocompromised persons, such as the aged, organ transplant recipients and persons infected with human immunodeficiency virus (HIV). CMV may also be associated with some cancers such as glioma. The invention therefore provides pharmaceutical compositions and/or methods of prophylactic or therapeutic treatment of CMV infection, preferably in humans.

Accordingly, in a related aspect, the invention provides a method of eliciting an immune response in a subject, said method including the step of administering to the subject a therapeutically effective amount of the pharmaceutical composition described herein, to thereby elicit the immune response in said subject.

Suitably, the method elicits or enhances an immune response in said subject to prevent or prophylactically or therapeutically treat a CMV-associated disease, disorder or condition, such as a CMV infection in the subject. More particularly, the administration of the pharmaceutical composition suitably elicits a protective immune response against CMV or a CMV infection in the subject.

In one embodiment, the immune response is or comprises a humoral immune response and/or a cell-mediated immune response, such as a cytotoxic T-lymphocyte immune response.

In a further aspect, a method of inducing immunity against a CMV infection in a subject, said method including the step of administering to the subject a therapeutically effective amount of the pharmaceutical composition of the first mentioned aspect to thereby induce immunity against the CMV infection in the subject.

The method of the present aspect suitably induces a protective immune response against the CMV infection. To this end, the immune response or immunity to the CMV or CMV infection suitably prevents the subject contracting a CMV-associated disease, disorder or condition.

In another embodiment, the present method suitably induces a humoral immune response and/or a cell-mediated immune response, such as a cytotoxic T-lymphocyte immune response, against the CMV infection.

In another aspect, the invention resides in a method of treating or preventing a CMV infection in a subject, said method including the step of administering to the subject a therapeutically effective amount of the pharmaceutical composition of the first mentioned aspect, to thereby prevent or treat the CMV infection in the subject.

As used herein, “treating” (or “treat” or “treatment”) refers to a therapeutic intervention that ameliorates a sign or symptom of a CMV infection, inclusive of a CMV-associated disease, disorder or condition, after it has begun to develop. The term “ameliorating,” with reference to a CMV-associated disease, disorder or condition, refers to any observable beneficial effect of the treatment. Treatment need not be absolute to be beneficial to the subject. The beneficial effect can be determined using any methods or standards known to the ordinarily skilled artisan.

As used herein, “preventing” (or “prevent” or “prevention”) refers to a course of action (such as administering a pharmaceutical composition of the present invention) initiated prior to the onset of a symptom, aspect, or characteristic of a CMV infection or a CMV-associated disease, disorder or condition, so as to prevent or reduce the symptom, aspect, or characteristic. It is to be understood that such preventing need not be absolute to be beneficial to a subject. A “prophylactic” treatment is a treatment administered to a subject who does not exhibit signs of a CMV infection or a CMV-associated disease, disorder or condition, or exhibits only early signs for the purpose of decreasing the risk of developing a symptom, aspect, or characteristic of a CMV infection or a CMV-associated disease, disorder or condition.

The term “therapeutically effective amount” describes a quantity of a specified agent, such as the pharmaceutical composition described herein, sufficient to achieve a desired effect in a subject being treated with that agent. For example, this can be the amount of a composition comprising the isolated proteins, the CMV envelope protein and the TLR agonist described herein, necessary to reduce, alleviate and/or prevent a CMV-associated disease, disorder or condition, inclusive of a CMV infection. In some embodiments, a “therapeutically effective amount” is sufficient to reduce or eliminate a symptom of a CMV infection or a CMV-associated disease, disorder or condition. In other embodiments, a “therapeutically effective amount” is an amount sufficient to achieve a desired biological effect, for example, an amount that is sufficient to elicit a protective immune response in a subject so as to inhibit or prevent a CMV infection.

Ideally, a therapeutically effective amount of an agent is an amount sufficient to induce the desired result without causing a substantial cytotoxic effect in the subject. The effective amount of an agent useful for reducing, alleviating and/or preventing a CMV-associated disease, disorder or condition, such as a CMV infection will be dependent on the subject being treated, the type and severity of any associated disease, disorder and/or condition (e.g., the type of CMV-associated disease, disorder or condition and/or strain of CMV), and the manner of administration of the therapeutic composition.

In the context of the present invention, by “CMV-associated disease, disorder or condition” is meant any CMV infection, inclusive of any clinical pathology resulting from such an infection by a cytomegalovirus, such as those hereinbefore described.

By “administering” or “administration” is meant the introduction of a composition disclosed herein into a subject by a particular chosen route. Any safe route of administration and dosage form, such as those hereinbefore described, may be employed for providing a patient with the composition of the invention.

As generally used herein, the terms “patient”, “individual” and “subject” are used in the context of any mammalian recipient of a treatment or composition disclosed herein. Accordingly, the methods and compositions disclosed herein may have medical and/or veterinary applications. In a preferred form, the mammal is a human.

In a further aspect, the invention resides in an isolated protein comprising each of the epitope amino acid sequences set forth in SEQ ID NOS: 1-20 or a fragment, variant or derivative thereof. Accordingly, the isolated protein may be or comprise an isolated polyepitope or polytope protein, such as that hereinbefore described.

In addition to the epitope amino acid sequences, the isolated protein may further comprise intervening amino acids or amino acid sequences, such as those described above. Suitably, the intervening amino acids or amino acid sequences are positioned or located relative to the epitope amino acid sequences to enable proteasomal processing and for transporting the proteasome-generated, individual epitope peptides into the endoplasmic reticulum (ER) for subsequent presentation with HLA-I molecules.

Furthermore, it will be appreciated that the particular arrangement or order of the constituent epitopes (i.e., SEQ ID NOs:1-20) of the isolated protein, such as that set forth in SEQ ID NO:21, may readily be altered while retaining broad HLA Class I-restricted immunogenicity.

It will also be appreciated that the isolated protein of the present aspect may include other epitope amino acid sequences in addition to those provided in SEQ ID NOs:1-20, such as those described in International Publication WO 03/000720 and International Publication No. WO2014/059489.

Suitably, the isolated protein comprises, consists essentially of, or consists of the amino acid sequence set forth in SEQ ID NO:21 or a fragment, variant or derivative thereof.

In particular embodiments, the isolated protein is for use in the methods previously described herein. To this end, the isolated protein may be suitable for inclusion in a pharmaceutical composition, such as that hereinbefore described.

The isolated protein of the present aspect, inclusive of fragments, variants and derivatives thereof, may be produced by any means known in the art, including but not limited to, chemical synthesis, recombinant DNA technology and proteolytic cleavage to produce peptide fragments.

In yet another aspect, the invention resides in an isolated nucleic acid encoding the isolated protein of the aforementioned aspect.

The term “nucleic acid” as used herein designates single-or double-stranded mRNA, RNA, cRNA, RNAi, siRNA and DNA inclusive of cDNA, mitochondrial DNA (mtDNA) and genomic DNA.

A “polynucleotide” is a nucleic acid having eighty (80) or more contiguous nucleotides, while an “oligonucleotide” has less than eighty (80) contiguous nucleotides. A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™. A “probe” may be a single or double-stranded oligonucleotide or polynucleotide, suitably labelled for the purpose of detecting complementary sequences in Northern or Southern blotting, for example.

In some embodiments, the isolated nucleic acid comprises, consists of, or consists essentially of the nucleotide sequence set forth in SEQ ID NO:22 or a fragment, variant or derivative thereof.

Also contemplated are fragments and variants of the isolated nucleic acid.

The invention also provides variants and/or fragments of the isolated nucleic acids. Variants may comprise a nucleotide sequence at least 70%, at least 75%, preferably at least 80%, at least 85%, more preferably at least 90%, 91%, 93%, 94%, 95%, 96%, 97%, 98% or 99% nucleotide sequence identity with any nucleotide sequence encoding the isolated protein of the invention (e.g., SEQ ID NO:22).

Fragments may comprise or consist of up to 5%, 10%, 15%, 20%, 25%, 30%, 35%, 40%, 45%, 50%, 55%, 60%, 65%, 70%, 75%, 80%, 85%, 90% or 95-99% of the contiguous nucleotides present in any nucleotide sequence disclosed herein.

Fragments may comprise or consist of up to 20, 50, 100, 150, 200, 250, 300, 350, 400, 450, 500, 550, 600, 650, or 660 contiguous nucleotides present in any nucleotide sequence disclosed herein.

The present invention also contemplates nucleic acids that have been modified such as by taking advantage of codon sequence redundancy. In a more particular example, codon usage may be modified to optimize expression of a nucleic acid in a particular organism or cell type.

The invention further provides use of modified purines (for example, inosine, methylinosine and methyladenosine) and modified pyrimidines (for example, thiouridine and methylcytosine) in isolated nucleic acids of the invention.

It will be well appreciated by a person of skill in the art that the isolated nucleic acids of the invention can be conveniently prepared using standard protocols such as those described in Chapter 2 and Chapter 3 of CURRENT PROTOCOLS IN MOLECULAR BIOLOGY (Eds. Ausubel et al. John Wiley & Sons NY, 1995-2008).

In yet another embodiment, complementary nucleic acids hybridise to nucleic acids of the invention under high stringency conditions.

“Hybridise and Hybridisation” is used herein to denote the pairing of at least partly complementary nucleotide sequences to produce a DNA-DNA, RNA-RNA or DNA-RNA hybrid. Hybrid sequences comprising complementary nucleotide sequences occur through base-pairing.

“Stringency” as used herein, refers to temperature and ionic strength conditions, and presence or absence of certain organic solvents and/or detergents during hybridisation. The higher the stringency, the higher will be the required level of complementarity between hybridizing nucleotide sequences.

“Stringent conditions” designates those conditions under which only nucleic acid having a high frequency of complementary bases will hybridize.

Stringent conditions are well-known in the art, such as described in Chapters 2.9 and 2.10 of Ausubel et al., supra, which are herein incorporated by reference. A skilled addressee will also recognize that various factors can be manipulated to optimize the specificity of the hybridization. Optimization of the stringency of the final washes can serve to ensure a high degree of hybridization.

Complementary nucleotide sequences may be identified by blotting techniques that include a step whereby nucleotides are immobilized on a matrix (preferably a synthetic membrane such as nitrocellulose), a hybridization step, and a detection step, typically using a labelled probe or other complementary nucleic acid. Southern blotting is used to identify a complementary DNA sequence; Northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al., supra, at pages 2.9.1 through 2.9.20. According to such methods, Southern blotting involves separating DNA molecules according to size by gel electrophoresis, transferring the size-separated DNA to a synthetic membrane, and hybridizing the membrane bound DNA to a complementary nucleotide sequence. An alternative blotting step is used when identifying complementary nucleic acids in a cDNA or genomic DNA library, such as through the process of plaque or colony hybridization. Other typical examples of this procedure are described in Chapters 8-12 of Sambrook et al., MOLECULAR CLONING. A Laboratory Manual (Cold Spring Harbor Press, 1989).

Methods for detecting labelled nucleic acids hybridized to an immobilized nucleic acid are well known to practitioners in the art. Such methods include autoradiography, chemiluminescent, fluorescent and colorimetric detection.

Nucleic acids may also be isolated, detected and/or subjected to recombinant DNA technology using nucleic acid sequence amplification techniques.

Suitable nucleic acid amplification techniques covering both thermal and isothermal methods are well known to the skilled addressee, and include polymerase chain reaction (PCR); strand displacement amplification (SDA); rolling circle replication (RCR); nucleic acid sequence-based amplification (NASBA), Q-β replicase amplification, recombinase polymerase amplification (RPA) and helicase-dependent amplification, although without limitation thereto.

As used herein, an “amplification product” refers to a nucleic acid product generated by nucleic acid amplification.

Nucleic acid amplification techniques may include particular quantitative and semi-quantitative techniques such as qPCR, real-time PCR and competitive PCR, as are well known in the art.

In still a further aspect, the invention provides a genetic construct comprising the isolated nucleic acid of the previous aspect.

In particular embodiments, the genetic construct comprises the isolated nucleic acid operably linked or connected to one or more other genetic components. A genetic construct may be suitable for therapeutic delivery of the isolated nucleic acid or for recombinant production of the isolated protein of the invention in a host cell.

Broadly, the genetic construct can be in the form of, or comprises genetic components of, a plasmid, bacteriophage, a cosmid, a yeast or bacterial artificial chromosome as are well understood in the art. Genetic constructs may be suitable for maintenance and propagation of the isolated nucleic acid in bacteria or other host cells, for manipulation by recombinant DNA technology and/or expression of the nucleic acid or an encoded protein of the invention.

For the purposes of host cell expression, the genetic construct is an expression construct. Suitably, the expression construct comprises the nucleic acid of the invention operably linked to one or more additional sequences in an expression vector. An “expression vector” may be either a self-replicating extra-chromosomal vector such as a plasmid, or a vector that integrates into a host genome.

By “operably linked” is meant that said additional nucleotide sequence(s) is/are positioned relative to the nucleic acid of the invention preferably to initiate, regulate or otherwise control transcription.

Regulatory nucleotide sequences will generally be appropriate for the host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, polyadenylation sequences, transcriptional start and termination sequences, translational start and termination sequences, and enhancer or activator sequences.

Constitutive, repressible or inducible promoters as known in the art are contemplated by the invention.

The expression construct may also include an additional nucleotide sequence encoding a fusion partner (typically provided by the expression vector) so that the recombinant protein is expressed as a fusion protein.

The expression construct may also include an additional nucleotide sequence encoding a selection marker such as amp^{R}, neo^{R} or kan^{R}, although without limitation thereto.

In particular embodiments, the expression construct may be in the form of plasmid DNA, suitably comprising a promoter operable in an animal cell (e.g. a CMV, an A-crystallin or SV40 promoter). In other embodiments, the nucleic acid may be in the form of a viral construct such as an adenoviral, vaccinia, lentiviral or adeno-associated viral vector.

In another aspect, the invention relates to a host cell transformed with a nucleic acid molecule or a genetic construct described herein.

Suitable host cells for expression may be prokaryotic or eukaryotic. For example, suitable host cells may include but are not limited to mammalian cells (e.g. HeLa, Cos, NIH-3T3, HEK293T, Jurkat cells), yeast cells (e.g. Saccharomyces cerevisiae), insect cells (e.g. Sf9, Trichoplusia ni) utilized with or without a baculovirus expression system, plant cells (e.g. Chlamydomonas reinhardtii, Phaeodactylum tricornutum) or bacterial cells, such as E. coli. Introduction of genetic constructs into host cells (whether prokaryotic or eukaryotic) is well known in the art, as for example described in CURRENT PROTOCOLS IN MOLECULAR BIOLOGY Eds. Ausubel et al., (John Wiley & Sons, Inc. 1995-2015), in particular Chapters 9 and 16.

Related aspects of the invention provide a method of producing the isolated protein described herein, including the steps of; (i) culturing the host cell of the previous aspect; and (ii) isolating said isolated protein from said host cell cultured in step (i).

In this regard, the recombinant protein may be conveniently prepared by a person skilled in the art using standard protocols, such as those hereinbefore provided.

In order that the invention may be readily understood and put into practical effect, particular embodiments will now be described by way of the following non-limiting examples.

### EXAMPLE

The present example investigated the immunogenicity of a vaccine formulation containing a polyepitope protein containing twenty (20) different epitopes derived from five different CMV antigens that cover a broad range of HLA molecules together with CMV glycoprotein B and the TLR9 agonist CpG 1018.

### Materials and Methods

CMVpoly20PL-NH Protein Expression and Purification Chemically competent E. coli BL21-codonPlus (DE3) RP cells (Agilent Technologies) were transformed with CMVpoly20PL-NH expression vector (pJ404-Atum Bio). Transformed cells were plated on Luria Bertani (LB) agar supplemented with ampicillin (LB-Amp) 100 m/mL and plates were incubated overnight at 37° C. An isolated colony was picked and inoculated into 10 ml of terrific broth containing 100 μg/mL ampicillin (TB-Amp broth) and grown in a shaker with 37° C. and 200 rpm for overnight. A small amount of overnight grown culture was inoculated into 50 mL of TB-Amp broth and grown for 12 hours. About 1% of culture from 50 mL culture transferred into 3 L of TB-Amp broth and culture was grown until OD reached to 0.6 at 600 nm. CMVpoly20PL-NH induction was carried out by adding 1 mM/mL of IPTG. These cells were allowed to grow for an additional 4 hours and protein expression levels were determined by analysing un-induced and induced samples on the 12% sodium dodecyl sulphate polyacrylamide gel electrophoresis (SDS-PAGE).

At the end of the induction phase, E. coli culture was harvested by centrifugation at 13,000 rpm for 15 minutes, cell pellet was resuspended in 100 mL of lysis buffer (25 mM Tris pH 7.5, 5 mM EDTA, 0.5% TritonX 100, 0.5 mg/mL lysozyme) supplemented with a protease inhibitor cocktail (Roche, Mannheim, Germany) and incubated on the ice for 30 minutes followed by cell lysis using sonicator. The sonication was carried out on ice for 6×8 minutes cycles (1 second on and off) with 10 minutes break between each cycle. The lysate was centrifuged at 13,000 rpm for 30 minutes and supernatant and pellet fractions were analysed on the SDS-PAGE gel.

Following SDS-PAGE analysis protein was found to be in the pellet in the form of inclusion bodies (IBs) and approximately 2 grams of (wet weight) pellet was obtained from every 3 L of induced culture. All the IBs washing, solubilisation and purification stages were carried out in the cold room. To eliminate the host protein contaminants, approximately 500 milligrams of IBs were washed with 2×600 mL of TE buffer (25 mM Tris and 5 mM EDTA pH 7.5-3×200 mL washes). To make homogenous suspension, IBs were suspended in TE buffer, sonicated for 10 minutes (1 second on and off cycles) and then solution was incubated at 4° C. under stirring for 30 minutes. At the end of every wash solution was centrifuged at 13,000 rpm for 30 minutes. Following TE buffer wash, IBs were washed with 2×200 mL of 100 mM NaH2PO4, 10 mM Tris, 4 M urea pH 7.5 buffer as mentioned above. Following centrifugation supernatant obtained from all the washes was analysed on SDS-PAGE gel to check CMVpoly20PL-NH protein loss. Finally, IBs were solubilised in 300 mL of 100 mM NaH2PO4, 10 mM Tris, 2.5 mM DTT, 8 M urea pH 5.5 buffer under stirring for 48 hours at 4° C. The soluble protein was clarified by centrifugation at 13,000 rpm for 30 minutes and all the samples were analysed on SDS-PAGE gel. The clarified supernatant was used for CMVpoly20PL-NH purification.

To purify the CMVpoly20PL-NH protein, 7 mL of SP-Sepharose matrix (GE healthcare) was washed with 10 column volumes of distilled water, 5 column volumes of 1 M NaOH, neutralised with distilled water, regenerated with 5 column volumes of 1 M NaCl and then equilibration with 5 column volumes of 100 mM NaH₂PO₄, 10 mM Tris, 2.5 mM DTT, 8 M urea pH 5.5 buffer. The soluble protein was mixed with equilibrated matrix in a 500 mL Duran bottle for 45 minutes at 4° C. Then slurry was transferred to the column and FT was collected. The unbound protein and impurities were washed-out with 20 column volumes of wash buffer; 100 mM NaH₂PO₄, 10 mM Tris, 2.5 mM DTT, 8 M urea pH 6.5. To elute bound protein, column was loaded with elution buffer; 100 mM NaH₂PO₄, 10 mM Tris, 2.5 mM DTT, 8 M urea pH 7.5 and incubated for 2 hours and then protein was eluted in 6×7 mL fractions. Again column was loaded with elution buffer; 100 mM NaH₂PO₄, 10 mM Tris, 2.5 mM DTT, 8 M urea pH 7.5 and incubated for another 2 hours and protein was eluted in another 6×7 mL fractions. Samples obtained from all the stages of purification were analysed on SDS-PAGE. All the purified protein fractions were pooled together and concentrated to a final volume of 50 mL.

To eliminate the host DNA and endotoxin contamination from the purified CMVpoly20PL-NH protein, HiSereen Q-FF Sepharose (5 mL-GE healthcare) was washed with 10 column volumes of distilled water, 5 column volumes of 1 M NaOH, neutralised with distilled water, regenerated with 5 column volumes of 1 M NaCl and then equilibration with 5 column volumes of 100 mM NaH₂PO₄, 10 mM Tris, 2.5 mM DTT, 8 M urea pH 7.5 buffer. Concentrated CMVpoly20PL-NH protein was loaded on the Q-FF and flow through was collected. To recover un-eluted protein, column was washed with 2 column volumes of 100 mM NaH₂PO₄, 10 mM Tris, 2.5 mM DTT, 8 M urea pH 7.5 buffer. All the samples at different stages of purification were analysed on the SDS-PAGE gel. Following SDS-PAGE analysis, flow through and wash were combined together; pH of the protein sample was decreased from 7.5 to 3.8. CMVpoly20PL-NH protein was dialysed against 25 mM glycine buffer at pH 3.8 using SnakeSkin dialysis tubing 10000 MWCO (Thermo Scientific, Rockford, USA) at 4° C. overnight. Following dialysis protein was concentrated and passed through Mustang E membrane (PALL Corporation, NY, USA) to further remove endotoxin contaminants, protein was filter sterilised using 0.22 μm membrane filter and then total protein was estimated using BIO-RAD Bradford protein assay kit and UV absorbance at 280 nm and purified protein was stored at −70° C.

### Protein Intact Mass Analysis.

Protein intact mass analysis was carried out at Australian Proteome Analysis Facility. In brief, protein samples were diluted to a concentration approximately 0.25 μg/μL with 2% acetonitrile and 0.1% formic acid and then sample (5 μL) was injected onto the column (Waters,)(Bridge BEH300 C4 3.5 μm 2.1×100 mm). Proteins was desalted on the column and was eluted from the column using a linear solvent gradient (A: 99.9% water+0.1% formic acid; B: 99.9% acetonitrile+0.1% formic acid). The protein electrospray-ionization mass spectra were averaged over the protein LC elution profile. The multiply charged protein spectrum was de-convoluted by Thermo Protein Deconvolution 2.0 software.

### In Vitro Immunogenicity Evaluation.

To determine the immunogenicity of CMVpoly20PL-NH protein, log phase T1 cells expressing human leucocyte antigen A2 (HLA A2) were washed with RPMI medium containing no serum and then pulsed with 25 μg of CMVpoly20PL-NH protein for one hour. CMVpoly20PL-NH pulsed T1 cell were washed and then incubated overnight in RPMI containing 5% FCS. Following overnight incubation cell were washed and exposed to CMV HLA A2 restricted epitope NLVPMVATV-specific CD8+ T cells at 1 CD8+ T cell: 1 APC and 4 CD8+ T cells: 1 APC ratios and incubated for four hours. Intracellular IFN-g expression was determined by ICS analysis. T1 cells pulsed with and without NLVPMVATV peptide used as positive and negative controls.

### Flow Cytometry Analysis.

Intracellular cytokine staining (ICS) assays were done using splenocytes to assess IFN-γ and multiple cytokines. Germinal centre B cell responses were determined by FACS analysis. Following vaccination, splenocytes were stimulated with 0.2 μg/mL of HLA matching CMV CD8+ T cell peptides (HLA A1-VTE & YSE; HLA A2-NLV, VLE & YIL; HLA A24-QYD & AYA; HLA B8-QIK, ELR & ELK; HLA B35-FPT & IPS) to determine the CMV-specific CD8+ T cell response or with 0.2 μg/mL of gB pepmix (gB overlapping peptides-15mers with 11 aa overlap) to detect CMV-specific CD4+ T cell response in the presence of GolgiPlug® (BD PharMingen) for 6 hours, cells were washed twice, then incubated with APC-conjugated anti-CD3, FITC-conjugated anti-CD4 and PerCP conjugated anti-CD8. Cells were fixed and permeabilised using a BD Cytofix/Cytoperm kit, then incubated with PE conjugated anti-IFN-γ. To assess the expression of multiple cytokines, cells were stained with PerCP conjugated anti-CD8 and BV786 anti-CD4 surface markers and then intracellularly with PE-conjugated anti-IFN-γ, PE-Cy7 conjugated anti-TNF, FITC conjugated CD017a and APC conjugated anti-IL2. To assess the germinal centre B cell response splenocytes or cells from lymph nodes from vaccinated mice were stained with PE conjugated anti-B220, FITC conjugated anti-GL7 and APC conjugated anti-CD95. Cells were acquired on a BD FACSCanto II and data was analysed using FlowJo software (Tree Star). Analysis of CMV-specific CD4+ and CD8+ T cells producing multiple cytokines was performed using Simplified Presentation of Incredibly Complex Evaluations (SPICE) software version 5.1.

Evaluation of Immunogenicity of CMV Vaccine Formulated with Various Combinations of Adjuvants.

All mouse studies were approved by QIMR Berghofer animal ethics committee. All human HLA transgenic mice (HLA A1, HLA A2, HLA A24, HLA B8 and HLA B35) were bred and maintained under pathogen-free environment at the QIMR Berghofer. These transgenic mice are deficient in expressing mouse MHC class I molecule and contain transgenes of the commonly expressed human HLA class I molecules. In order to evaluate the immunogenic response to the CMVpoly20PL-NH polypeptide and CMV glycoprotein B (gB), proteins were formulated with three different forms of Monophosphoryl Lipid A (Synthetic) (Avanti Polar Lipids) and unmethylated CpG oligodeoxynucleotides 1018 (CpG 1018) (TriLink BioTechnologies) adjuvants. Six to eight week old mice were immunized subcutaneously (s.c.) with 100 μL volume at the base of the tail with CMV vaccine formulation containing 30 μg of CMVpoly20PL-NH and 5 μg of gB protein formulated with 25 μg of PHAD and 50 μg of CpG 1018, 25 μg of 3D-PHAD and 50 μg of CpG 1018, 25 μg of 3D(6-acyl)-PHAD and 50 μg of CpG 1018 or 50 μg of CpG 1018 alone. Mice immunised with 30 μg of CMVpoly20PL-NH and 5 μg of gB protein formulated with 25 μg of MPL and 50 μg of CpG 1826 or adjuvants alone is used as positive and negative controls. Mice were tail bled on day 21 and a booster dose was given. Mice were then sacrificed in day 28 and evaluated for immune responses. Control group mice were injected with adjuvants alone. To assess the long-term durability of CMV vaccine induced immune responses HLA A24 and HLA A1 human HLA transgenic mice were immunised with CMV vaccine formulation containing 30 μg of CMVpoly20PL-NH and 5 μg of gB protein formulated with 50 μg of CpG 1018 on day 0 and then boosted on day 21, 42 and 210. Control group mice were injected with CpG1018 adjuvant alone. Mice were sacrificed on day 28, 42, 49, 84, 133, 203 and 217 to evaluate long-term CMV-specific immune responses.

### Intracellular Cytokine Staining to Assess IFN-γ, Multiple Cytokine, Germinal Centre B Cells or T Follicular Helper Cells Responses

Following vaccination, splenocytes were stimulated with 0.2 μg/mL of HLA matching CMV CD8+ T cell peptides (HLA A1-VTE & YSE; HLA A2-NLV, VLE & YIL; HLA A24-QYD & AYA; HLA B8-QIK, ELR & ELK; HLA B35-FPT & IPS) to determine the CMV-specific CD8+ T cell response or with 0.2 μg/mL of gB pepmix (gB overlapping peptides-15mers with 11 aa overlap) to detect CMV-specific CD4+ T cell response in the presence of GolgiPlug® (BD PharMingen) for 6 hours, cells were washed twice, then incubated with APC-conjugated anti-CD3, FITC-conjugated anti-CD4 and PerCP conjugated anti-CD8. Cells were fixed and permeabilized using a BD Cytofix/Cytoperm kit, then incubated with PE conjugated anti-IFN-γ. To assess the expression of multiple cytokines, cells were stained with PerCP conjugated anti-CD8 and BV786 anti-CD4 surface markers and then intracellularly with PE-conjugated anti-IFN-γ, PE-Cy7 conjugated anti-TNF, FITC conjugated CD017a and APC conjugated anti-IL2. To assess the germinal centre B cell response splenocytes or cells from lymphnodes from vaccinated mice were stained with PE conjugated anti-B220, FITC conjugated anti-GL7 and APC conjugated anti-CD95. Cells were acquired on a BD FACSCanto II and data was analysed using FlowJo software (Tree Star).

### In Vitro Expansion of CMV-Specific CD4+ and CD8+ T Cells Following Vaccination

Following vaccination, 5×10⁶ Splenocytes from immunized mice were isolated and stimulated with 0.2 μg/mL of matching CMV CD8+ T cell peptides or gB pepmix (gB overlapping peptides-15mers with 11 aa overlap) and cell were cultured in a 24 well plate for 10 days at 37° C. 10% CO₂ Cultures were supplemented with recombinant IL-2 on days 3 and 6 and on day 10 and T cell specificity was assessed using ICS assay.

### Mouse IgG ELISpot Assay

To measure ex vivo gB-specific antibody secreting cells, PVDF ELISpot plates (Millipore) were treated with 70% ethanol. Plates were washed five times with distilled water, coated with 100 μL/well CMV gB protein (25 μg/mL) or anti-IgG antibody (15 μg/mL) and incubated overnight at 4° C. Plates were blocked with DMEM containing 10% serum, 300,000 cells/well in triplicates from each mouse was added and then incubated for 18 hours in a 37° C. humidified incubator with 5% CO₂. Cells were removed and plates were washed. Detection antibody anti-IgG conjugated to HRP (MABTECH) was added and incubated for 2 hours at room temperature. Plates were washed; Streptavidin-ALP was added and incubated at room temperature for 1 hour followed by washing and treating plates with substrate solution containing BCIP/NBT (Sigma-Aldrich) until colour development is prominent. Colour development was stopped by washing plates with water and plates were kept for drying overnight. To measure memory B cell response, the spleen cells (5×10⁵) were activated with a mixture of R484 and recombinant mouse IL-2 for five days in 24 well plate and then ELISpot was carried out as stated above. Number of spots were counted in an ELISpot reader.

### ELISA

Serum total anti-gB antibody and antibody isotype titres were evaluated by an enzyme-linked immunosorbent assay (ELISA). Briefly, 96-well plates pre-coated with 50 μL of recombinant HCMV gB protein (2.5 μg/mL of gB protein diluted in carbonate buffer) and plates were incubated at 4° C. o/n. Plates were washed with phosphate buffer saline containing 0.05% Tween 20 (PBST) buffer and then blocked with 5% skim milk. The serially diluted serum samples (day 21 or day 28) were added and incubated for 2 hours at room temperature. After washing with PBST, plates were incubated with HRP-conjugated sheep anti-mouse Ig antibody (to determine total antibody response) or HRP-conjugated goat anti-mouse IgA, IgM, IgG1, IgG2a, IgG2b or IgG3 antibody (SouthernBiotech) (to determine antibody isotype) for 1 hour. These plates were washed and incubated with 3,3′,5,5′-Tetramethylbenzidine substrate solution for 10 mins and then colour development was stopped by adding 1 N HCl. OD at 450 nm was measured using an ELISA reader.

### Microneutralisation Assay

Neutralizing activity was determined against AD169 and TB40/E strains of CMV. Human fibroblast MRC-5 or Adult Retinal Pigment Epithelial (ARPE-19) cells were plated in 96-well flat-bottomed plates. The next day, serum samples from mice vaccinated with CMV vaccine formulation were serially diluted and added to a standard number of virus particles (1000 p.f.u. per well) diluted in DO (DMEM with no serum) in 96-well U-bottomed plates and incubated for 2 h at 37° C. and 5% CO₂. As a positive control, virus without serum and a negative-control serum without virus were also included in the test. The serum/CMV mixture was then added to the MRC-5 or ARPE-19 cells and incubated at 37° C. and 5% CO₂ for 2 hours. After incubation, the mixture was discarded and the cells washed gently five times with DMEM containing 10% FCS (D10) and a final volume of 200 ml R10 was added to each well, followed by incubation for 16-18 hours at 37° C. and 5% CO2. The cells were fixed with 100 mL chilled methanol and incubated with Peroxidase Block (Dako) followed by mouse anti-CMV IE-1/IE2 mAb (Chemicon) at room temperature for 3 hours. Cells were then incubated with 50 ml HRP-conjugated goat anti-mouse Ig (diluted 1:200 in PBS) per well for 3 hours at room temperature. The cells were stained with 20 μL diaminobenzidine plus substrate (Dako) per well for 10 min at room temperature and positive nuclei that stained dark brown were counted. The neutralizing titre was calculated as the reciprocal of the serum dilution that gave 50% inhibition of IE-1/IE-2-expressing nuclei.

CMV gB-Specific Antibodies Binding to Cell-Associated gB on CMV-Infected Fibroblasts Assay.

Human fibroblasts cell line, MRC-5 cells were grown to 50% confluency in a T75 flask. Cell were infected with CMV AD169 strain at multiplicity of infection (MOI) of 2.0 at 37° C. and 5% CO₂ for 2 hours. Following infection cells were washed and incubated with DMEM containing 10% FCS for 48 hours to allow cell-cell virus spared. Infected cells were washed with PBS and then cells were dislodged with trypsin-EDTA. Cells were washed, counted and then resuspended at 10⁶ viable cells/mL. Cells were stained with cell trace violet and incubated for 20 minutes at room temperature. Cells were washed and then fixed with 4% paraformaldehyde for 10 minutes at room temperature. Cells were washed twice, plated 20,000/well in 96-well V-bottom plates, cells were pelleted by centrifugation and supernatant was discarded. Mouse serum samples obtained from HLA A24 human transgenic mice following immunisation with CMV vaccine or place on day 21, 28 42, 49, 84, 133, 203 and 217 were polled and then diluted to 1:512. Diluted serum samples were added to the MRC-5 cells and incubated for 2 hours at 37° C. and 5% CO₂. Cells were washed and then stained with anti-mouse AF488 IgG (H+L) for 30 minutes at 4° C. Cells were acquired on a BD FACSCanto II and data was analysed using FlowJo software (Tree Star). The percentage of CMV gB-specific antibody binding to CMV infected fibroblasts was calculated from the percentage of viable AF 488 positive cells.

### Results

### CMVpoly20PL-NH Protein Constructs Design, Protein Expression, Purification Process Development, Protein Characterisation and In Vitro Immunogenicity Evaluation

CMV-specific immune responses are not restricted to gB, pp65 and IE-1 antigens as previously understood, but are directed towards more than 70% of the CMV reading frames (Elkington, Walker et al. 2003, Elkington, Shoukry et al. 2004, Manley, Luy et al. 2004, Sylwester, Mitchell et al. 2005). Therefore, a vaccine which can induce a broad repertoire of virus-specific immune responses is likely to provide more effective protection against virus-associated pathogenesis. Thus, to target broad repertoire of immunodominant CMV antigens, the inventors designed a novel multivalent vaccine consisting of CMV glycoprotein B (gB) and polyepitope (CMVpoly20PL-NH) recombinant proteins with human compatible adjuvant(s) because multivalent vaccines likely to provide more long-lived immunity than monovalent vaccines. The CMV gB used in this vaccine formulation consisting of extracellular and intracellular domains and it is a major target for neutralising antibody and CD4+ and CD8+ T cell responses. Another protein is the CMVpoly20PL-NH designed to encode multiple HLA class I restricted CD8+ T-cell epitopes from five highly conserved immunodominant antigens (pp65, IE-1, pp150, pp50 and DNAse) of CMV. The polyepitope sequence was designed in such a way that at carboxyl terminus of each epitope was joined by a proteasome liberation amino acid sequence (AD or K or R) (Table 1). In our previous studies, we have shown that proteasome liberation amino acid sequence improves the immunogenicity of CD8+ T cell epitopes by enhancing proteasomal processing of the polyepitope protein (Dasari, Smith et al. 2014). The amino acid sequence of CMVpoly20PL-NH construct was translated into DNA sequence based on E. coli codon utilization (FIG. 1) and CMV polyepitope encoding sequence was synthetically constructed and cloned into an expression plasmid under an isopropyl-β-D-thiogalactopyraniside (IPTG) inducible promoter. The synthetically designed CMVpoly20PL-NH construct was transformed into chemically competent E. coli DH5a cells and plasmid was subsequently isolated and purified.

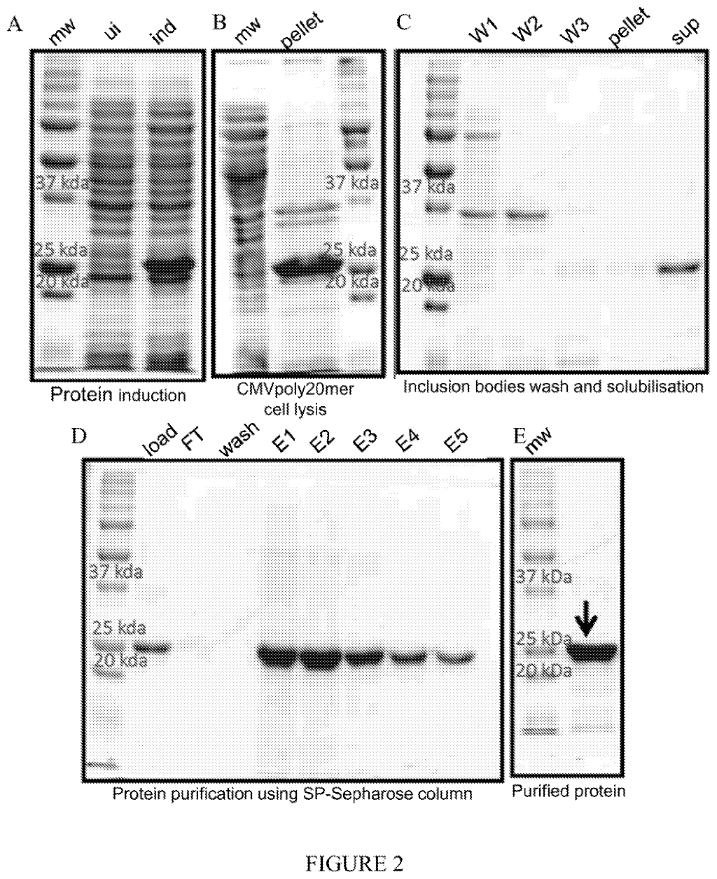

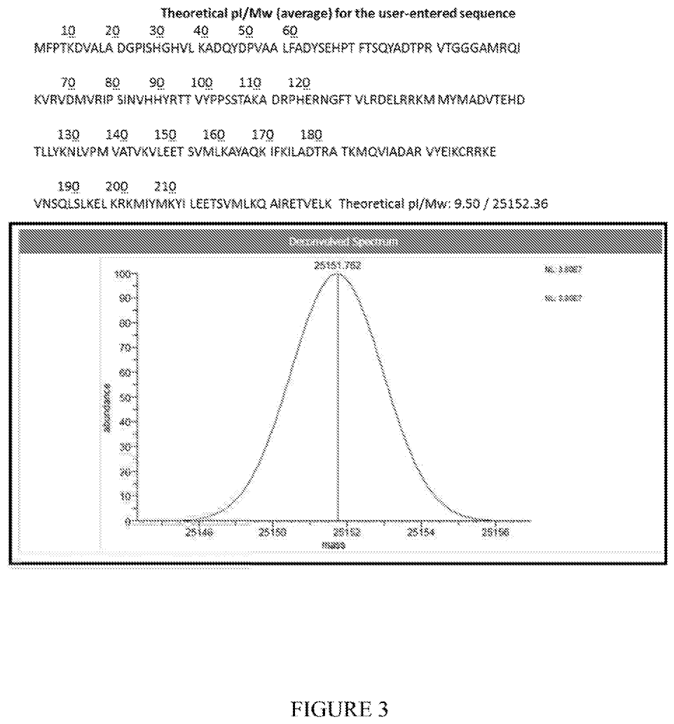

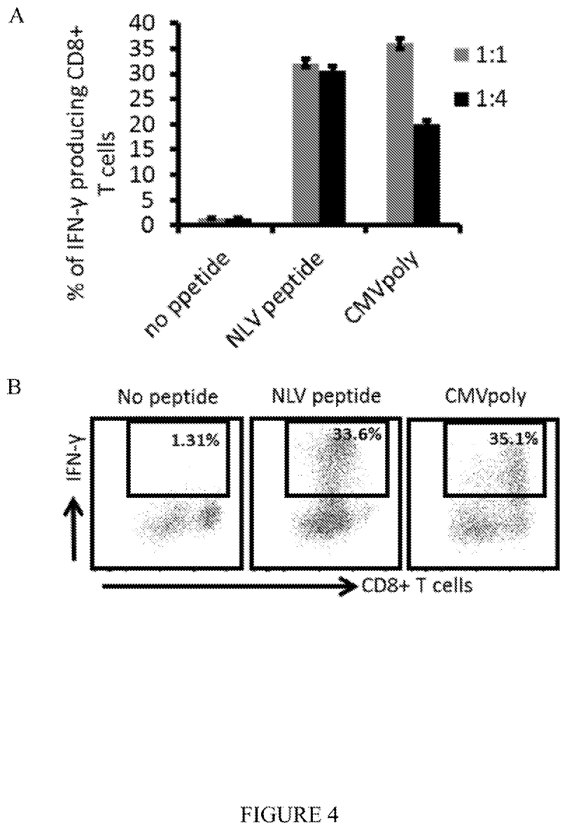

CMVpoly20PL-NH expression vector was transformed into E. coli BL21-codonPlus (DE3) RP protein expression host. Following transformation of the CMVpoly20PL-NH expression vector into E. coli, the expression of CMVpoly20PL-NH protein was induced using IPTG. At the end of the induction phase, E. coli culture was harvested and analysed to determine the protein expression levels. Data obtained from this analysis showed that CMVpoly20PL-NH expression vector produced high levels of CMVpoly20PL-NH protein (FIG. 2A). To purify CMVpoly20PL-NH protein cell lysis was carried and the supernatant and pellet fractions from cell lysate was analysed using SDS-PAGE. Due to the high hydrophobic nature of the linear CD8+ T cell epitopes, the CMVpoly20PL-NH protein was aggregated in the form of inclusion bodies (IBs) (FIG. 2B). To eliminate host cell proteins and DNA contamination, IBs were washed with TE buffer (FIG. 2C) and then solubilised. The soluble protein was purified using SP-Sepharose column in batch mode, eluted CMVpoly20PL-NH protein from SP-Sepharose column was loaded on Q-Sepharose column to eliminate host DNA and endotoxin contaminants and then purified CMVpoly20PL-NH protein was dialysed against 25 mM glycine pH3.8 buffer and then all the samples were analysed on SDS-PAGE (FIGS. 2D and E). To further eliminate residual endotoxin contaminants dialysed protein was filtered through Mustang E™ 0.22 μm membrane. Data obtained from these experiments shows that CMVpoly20PL-NH protein could be successfully expressed and purified to homogeneity using a bacterial expression system. To confirm the identity of CMVpoly20PL-NH protein, we performed protein intact mass analysis and the mass of the CMVpoly20PL-NH of the purified polypeptide is consistent with the theoretical molecular weight predicted by the amino acid sequence (FIG. 3). To determine the immunogenicity of the CMVpoly20PL-NH protein, human lymphoblastoid cell lines (LCLs) were pulsed overnight with CMVpoly20PL-NH protein and then assessed their ability to activate CMV-specific CD8+ T cells using intracellular IFN-γ analysis. The HLA A2-restricted pp65 epitope, NLVPMVATV (referred to as NLV), from CMVpoly20PL-NH was more efficiently processed and presented to NLV-specific CD8+ T cells compared with LCLs pulsed with LCLS pulsed with NLV epitope (FIG. 4).

### CMV Vaccine Formulation Development and Immunogenicity Evaluation in Human HLA A24 Transgenic Mice

Recent understandings in the immunology, pathology, and molecular biology of CMV have suggested that protection against CMV-related disease is mediated by both humoral and cellular immunity, thus, an ideal vaccine against CMV needs to induce both humoral and cellular responses. Unfortunately, recent attempts to develop a CMV vaccine have demonstrated limited success. These CMV vaccine strategies have assessed glycolprotein B (gB), pp65 and IE-1 as potential targets and they have been delivered by numerous delivery platforms, including the attenuated CMV Towne strain (Jacobson, Sinclair et al. 2006) recombinant viral vectors encoding full length antigens and epitopes (Bernstein, Reap et al. 2009, Zhong and Khanna 2009, La Rosa, Longmate et al. 2017), DNA (Wloch, Smith et al. 2008) dense body (Pepperl-Klindworth, Frankenberg et al. 2002), subunit (Drulak, Malinoski et al. 2000) vaccines and most recently a conditionally replication-defective CMV vaccine derived from AD169 strain (Adler, Lewis et al. 2019). Over the years it has been believed that in order to elicit a protective, CD8 cytotoxic T cell response, viral antigens must be delivered in nucleic acid form (e.g., using a viral vector delivery system or DNA plasmids) rather than as exogenously-delivered proteins, so that the expressed polypeptide is properly processed and presented to T cells. However, the majority of these vaccine delivery platforms, in particular live-attenuated vaccines and viral vector-based vaccines, have raised several regulatory concerns, such as perceived long-term theoretical health risks and pre-existing immunity (Lee, Markham et al. 2012). To overcome immunogenicity and safety issues, we have developed a novel multivalent vaccine platform technology using human compatible adjuvant system to generate a CMV-specific neutralising antibody response, CD4+ and CD8+ T cells responses against six antigens expressed in different stages of CMV infection and replication.

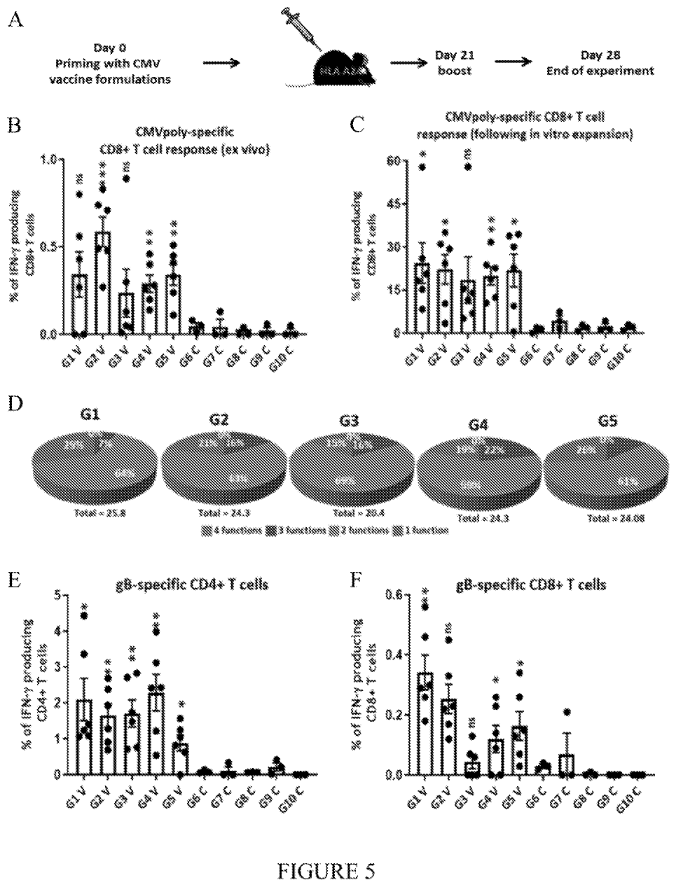

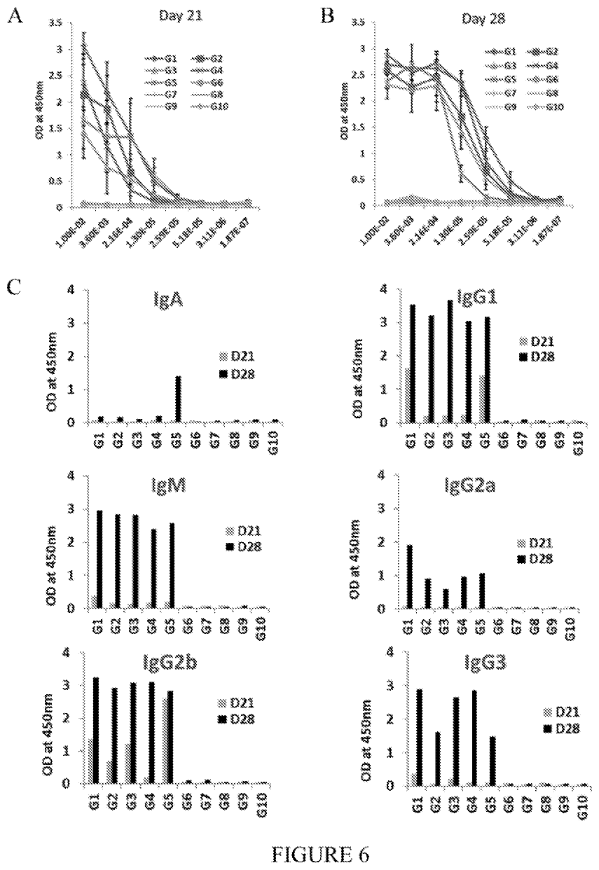

To generate robust immune response against CMV gB and CMVpoly20PL-NH proteins, we have formulated these two proteins with CpG 1018 alone or with three different forms of MPLA (PHAD, 3D-PHAD and 3D(6-acyl)-PHAD. Human HLA 24 transgenic mice were immunised with different CMV vaccine formulations on day 0 and these mice were tail bleed on day 21 and boosted with an identical formulation. Immunised mice were sacrificed to evaluate CMV-specific immune responses on day 28 (FIG. 5A). Immunisation with the CMV gB and CMVpoly20PL-NH proteins, formulated with PHAD and CpG 1018, 3D-PHAD and CpG 1018, 3D(6-acyl)-PHAD, CpG 1018 alone or MPL and CpG 1826 resulted a strong ex vivo IFN-γ secreting CMV-specific CD8+ T cell responses (specific to HLA A24 restricted QYD and AYA peptides) compared to adjuvant alone control groups (FIG. 5B). However, a significant enhancement in IFN-γ secreting CD8+ T cell responses were observed in mice immunised with CMV gB and CMVpoly20PL-NH formulated with 3D-PHAD and CpG 1018 or CpG 1018 alone (FIG. 5B). In addition, due to low CD8+ T cell numbers in human HLA transgenic mice, we further confirm the immunogenicity of all vaccine formulations by expanding vaccine induced CMV-specific CD8+ T cell ex vivo. Interestingly, following in vitro expansion at least 20-fold increase in IFN-γ producing CMV-specific CD8+ T cells were observed in mice immunised with CMV gB and CMVpoly20PL-NH proteins, formulated with PHAD and CpG 1018, 3D-PHAD and CpG 1018, 3D(6-acyl)-PHAD, CpG 1018 alone or MPL and CpG 1826 (FIG. 5C). In addition to IFN-γ secretion, we also assessed vaccine induced CMV-specific CD8+ T cells for their ability to secrete multiple cytokines because polyfunctional T cells play a crucial role in controlling CMV infection (Crough, Beagley et al. 2012, Gibson, Barysauskas et al. 2015). Ex vivo expansion of splenocytes from mice vaccinated with CMV gB and CMVpoly20PL-NH proteins, formulated with PHAD and CpG 1018, 3D-PHAD and CpG 1018, 3D(6-acyl)-PHAD, CpG 1018 alone or MPL and CpG 1826 resulted in substantial populations of CMV-specific CD8+ T cells expressing 2 or 3 mediators (IFN-γ, TNF or IL-2) (FIG. 5D). In the next set of experiments, mice were immunised with CMV gB and CMVpoly20PL-NH proteins, formulated with PHAD and CpG 1018, 3D-PHAD and CpG 1018, 3D(6-acyl)-PHAD, CpG 1018 alone or MPL and CpG 1826 and their ability to induce gB-specific antibody and CD4+ and CD8+ T cell responses were assessed. Ex vivo analysis shows that all the vaccine formulations induced strong CMV-gB specific CD4+ and CD8+ T cell responses compared to adjuvant alone controls; however, a significant enhancement of gB-specific CD4+ and CD8+ T cell responses were observed in mice vaccinated with CMV gB and CMVpoly20PL-NH proteins, formulated with 3D-PHAD and CpG 1018 or CpG 1018 alone (FIGS. 5E and 5F). Subsequently, the present inventors evaluated the gB-specific antibody response in serum samples of mice vaccinated with CMV gB and CMVpoly20PL-NH proteins, formulated with PHAD and CpG 1018, 3D-PHAD and CpG 1018, 3D(6-acyl)-PHAD, CpG 1018 alone, MPL and CpG 1826 or adjuvants alone. The gB and CMVpoly20PL-NH formulated with PHAD and CpG 1018, 3D-PHAD and CpG 1018, 3D(6-acyl)-PHAD, CpG 1018 alone or MPL and CpG 1826 to induced gB-specific antibody titres following the primary immunization (day 21) and these titres were further increased following a booster dose on day 28 in all groups compared to serum obtained from mice immunised with adjuvants alone (FIGS. 6A and 6B). The isotypes of anti-gB antibodies in serum samples were also evaluated. The combination of CMV gB CMVpoly20PL-NH formulated with PHAD and CpG 1018, 3D-PHAD and CpG 1018, 3D(6-acyl)-PHAD, CpG 1018 alone or MPL and CpG 1826 induced a CMV-gB-specific antibody response which included multiple isotypes, including IgM, IgG1 (Th2-like Ig isotype), and IgG2b, IgG2a, IgG3 (Th1-like Ig isotypes) (FIG. 6C). Collectively these results demonstrated that CMV gB and CMVpoly20PL-NH proteins formulated with either 3D-PHAD and CpG 1018 or CpG 1018 alone could generate a robust CMVpoly20PL-NH-specific CD8+ T cell response and gB-specific CD4+ and CD8+ T cell and antibody responses compared formulation with 3D-PHAD and CpG 1018 or 3D(6-acyl)-PHAD. Because there was no significant difference in immune responses generated against CMV gB and CMVpoly20PL-NH proteins when formulated with 3D-PHAD and CpG 1018 or CpG 1018 alone in the subsequent experiments we used CMV gB and CMVpoly20PL-NH proteins formulated with CpG 1018 alone to test immunogenicity and durability of CMV vaccine induced immune responses.

Extended Preclinical Assessment of CMV Vaccine Formulated with CpG 1018 Adjuvant in Multiple HLA Transgenic Mice

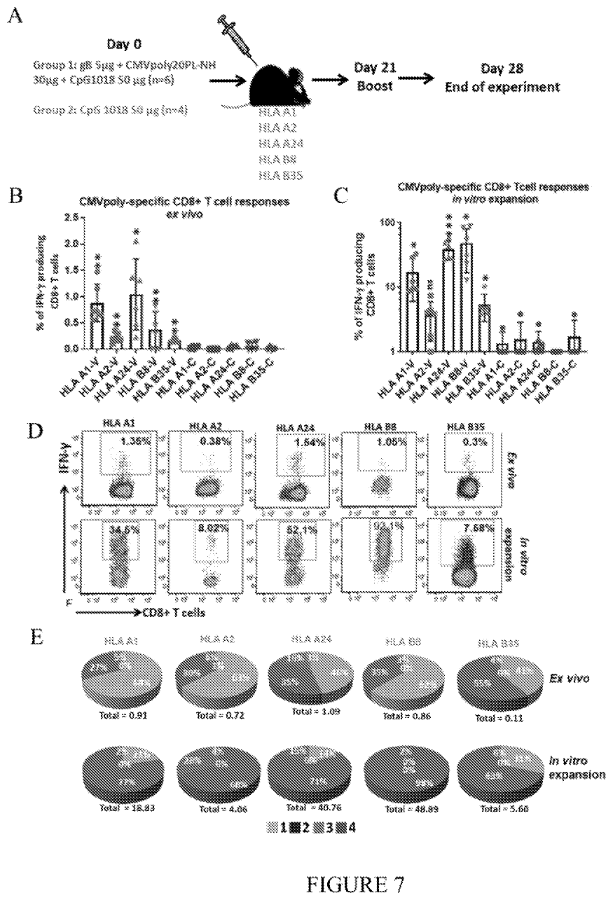

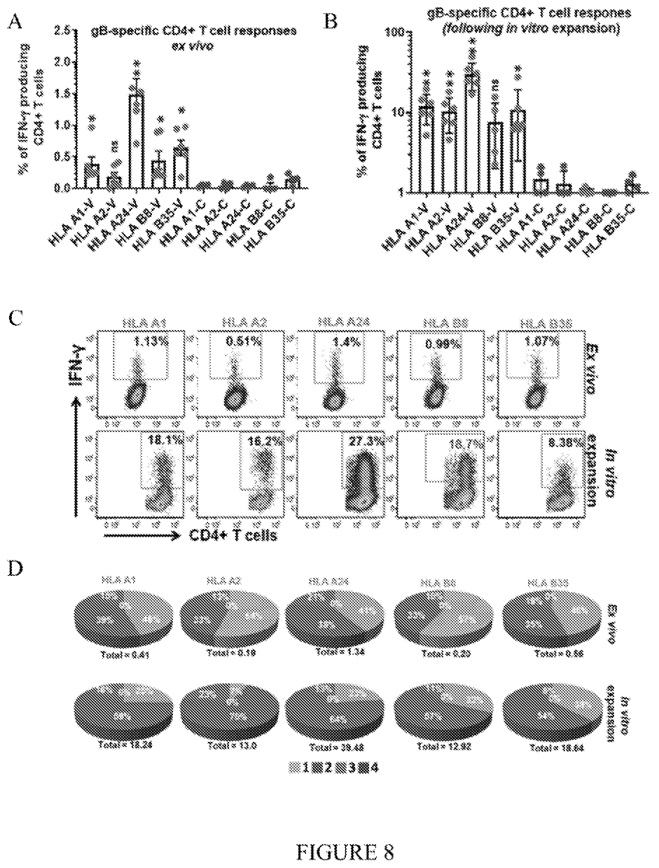

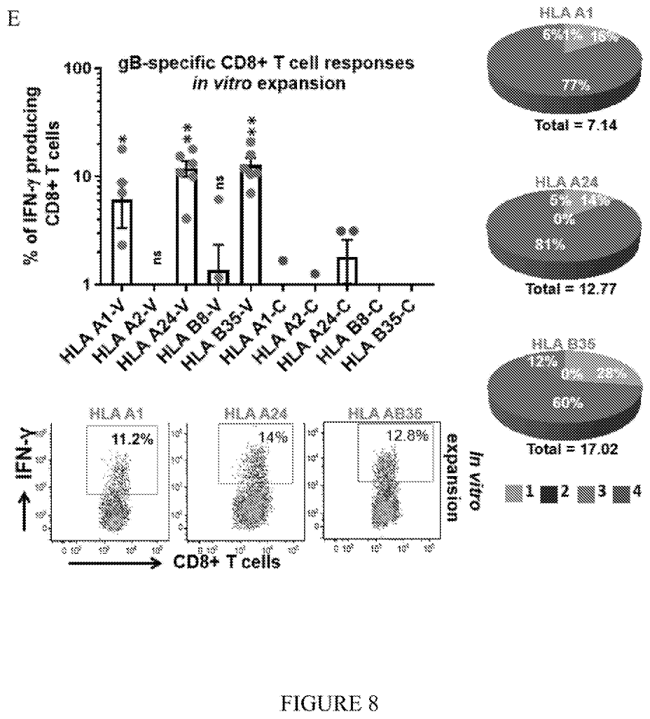

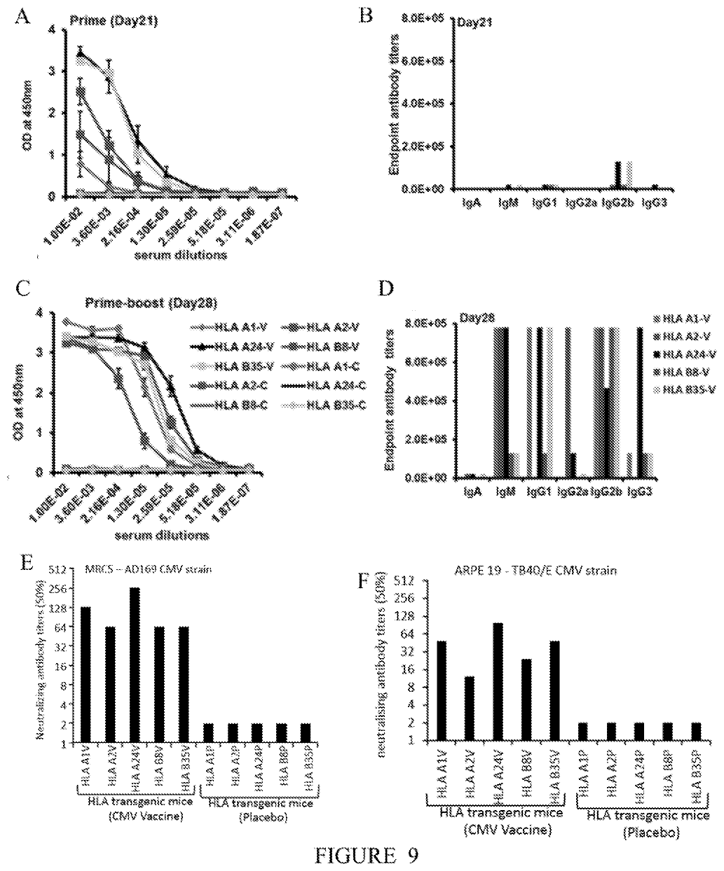

Although robust immune responses against CMV gB and CMVpoly20PL-NH proteins in human HLA A24 transgenic mice were observed, it is important to assess the immunogenicity of gB and CMVpoly20PL-NH in other human HLA transgenic mice. These multiple human HLA transgenic mice offer a greater advantage to evaluate immunogenicity of CMVpoly20PL-NH protein CD8+ T cell epitopes restricted to a variety of human HLA class I molecule. In order to assess the immunogenicity of CMV gB and CMVpoly20PL-NH proteins, human HLA A1, A2, A24, B8 and B35 transgenic mice were immunised with CMV gB and CMVpoly20PL-NH formulated with CpG 1018 alone (vaccine group) or CpG alone (control group) on day 0. Mice were tail bled and boosted on day 21 and sacrificed on day 28 (FIG. 7A). In consistent with the immunogenicity data seen in human HLA A24 transgenic mice, ex vivo analysis clearly revealed that the formulation of CMV gB and CMVpoly20PL-NH with CpG 1018 alone induced significantly higher CMV-specific CD8+ T cell responses (restricted to HLA A1, A2, A24, B8 and B35 epitopes) in all human HLA transgenic mice compared to mice immunised with CpG 1018 alone (FIGS. 7B and 7D). These ex vivo immune responses were further confirmed by in vitro stimulating vaccinated mice splenocytes with respective peptides and culturing them for ten days. Interestingly, ex vivo stimulation resulted a significant expansion of CMV-specific CD8+ T cell responses. Specifically, compared to ex vivo responses following in vitro stimulation a 40- to 200-fold increase in IFN-γ secreting CD8+ T cells were observed (FIGS. 7C and 7D). In addition, CMV gB and CMVpoly20PL-NH formulated with CpG 1018 alone also induced polyfunctional CMV-specific CD8+ T cells, with a range between 32% to 59% ex vivo and 69% to 98% in vitro expanded CMV-specific CD8+ T cells secrete at least two cytokines (IFN-γ, TNF or IL-2) (FIG. 7E). The CMV gB and CMVpoly20PL-NH formulated with CpG 1018 alone also induced significantly higher CMV-gB specific CD4+ T cells producing IFN-γ in HLA A1, A24, B8 and B35 mice compared to mice immunised with CpG 1018 alone (FIGS. 8A and 8C). Subsequent analysis following in vitro expansion with gB pepmix resulted 20- to 90-fold increase in expansion of CMV-gB specific CD4+ T cells producing IFN-γ in HLA A1, A2, A24, B8 and B35 transgenic mice compared to ex vivo responses (FIGS. 8B and 8C). Remarkably, ex vivo and in vitro expansion analysis showed that the CMV gB and CMVpoly20PL-NH formulated with CpG 1018 alone also induced polyfunctional gB-specific CD4+ T cell responses in all HLA transgenic mice, in particular at least 43% of ex vivo and 62% of gB-specific CD4+ T cells secrete at least two cytokines (IFN-γ, TNF or IL2) (FIG. 8D). Additionally, the CMV gB, CMVpoly20PL-NH and CpG 1018 formulation further demonstrated its ability to induce CMV gB-specific CD8+ T cell responses in HLA A1, A24 and B35 mice and a substantial proportion of these gB-specific CD8+ T cell were able to secrete two or more cytokines (FIG. 8E). To confirm whether the CMV gB, CMVpoly20PL-NH and CpG 1018 formulation also able to promote robust antibody responses in addition to strong CMV-specific cellular immune responses, we assessed serum anti-gB antibody titres using ELISA in all human HLA transgenic mice. The CMVpoly20PL-NH, gB and CpG 1018 formulation induced gB-specific antibody titres following the primary immunization (day 21) in all human transgenic mice but low antibody titres were observed in HLA A1 and B8 transgenic mice compared to HLA A2, A24 and B35 transgenic mice and the predominant antibody isotype after 21 days of primary dose was IgG2b (Th1-like Ig isotype) (FIGS. 9A and 9B). However, a booster dose on day 28 resulted a significant enhancement in gB-specific antibody titres in all human HLA transgenic mice (FIGS. 9A and 9B) and major antibody isotypes were Th2-like IgM and IgG1 and Th1 like IgG2b and IgG3. In the subsequent analysis, we tested the functionality of these antibodies in microneutralisation assay. Serum from all human HLA transgenic mice immunised with CMV gB, CMVpoly20PL-NH and CpG 1018 formulation induced strong neutralising antibody response against HCMV AD169 strain compared to mice immunised with CpG 1018 alone, however neutralising antibody titres in HLA A24 were dramatically higher than other human HLA transgenic mice (FIG. 9E).

The inventors then explored the ability of CMV vaccine induced neutralising antibody responses in all human HLA transgenic mice against a different CMV strains TB40/E in ARPE-19 cells (FIG. 9F). Although, overall neutralising antibody titres were lower compared to AD 169 strain, serum obtained from all human HLA transgenic mice following immunisation with CMV vaccine demonstrated strong neutralising activity against TB40/E strain compared to serum obtained from mice immunised with placebo. Interestingly, HLA A1, B35 and A24 mice demonstrated higher neutralising antibody titres compared to HLA A2 and B8 mice. Collectively, these observations suggest that CMV vaccine formulated with gB, CMVpoly20PL-NH and CpG 1018 able to induce strong neutralising antibody responses in multiple human HLA transgenic mice against multiple CMV strains (AD 169 and TB40/E), which is regarded as comprehensive approach in determining the CMV vaccine efficacy.

### Assessment of Long-Term Durable Immunity Induced by CMV gB, CMVpoly20PL-NH and CpG 1018 Vaccine Formulation

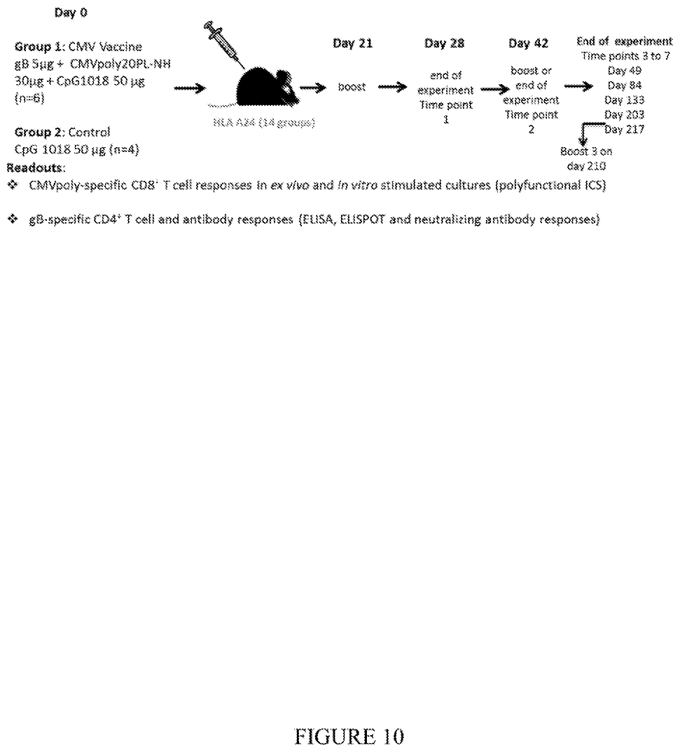

One of the major problems with previous CMV vaccines is the durability of vaccine induced immunity and low efficacy. In fact, durable vaccine-induced immune responses play a crucial role in minimising waning efficacy of vaccines against diverse pathogens (Amanna, Carlson et al. 2007). Therefore, it is important to test the long-term durability of CMV gB, CMVpoly20PL-NH and CpG1018 vaccine formulation induced immune responses and number of booster doses required to maintain persistent immune responses against CMV. In order to test the long-term durable immunity, we immunised human HLA A24 transgenic mice with CMV gB, CMVpoly20PL-NH and CpG1018 vaccine formulation (vaccine group) or CpG 1018 alone (control group) on day 21, boosted on day 28 and 42. To recall the vaccine induced pre-existing CMV-specific immune response a sub-group of mice were given a third boost on day 210. We monitored CMV-specific immune responses in vaccinated human HLA A24 transgenic mice by longitudinally (day 28, 42, 48, 84, 133, 203 and 217) measuring CMVpoly20PL-NH-specific CD4+ T cell responses, gB-specific CD4+ and CD8+ T cell, B cell and antibody responses (FIG. 10).

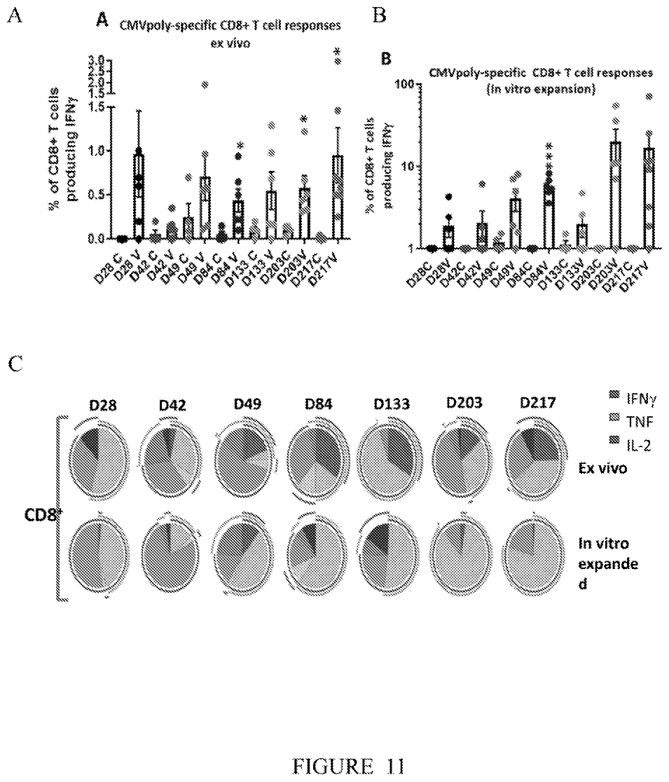

Ex vivo analysis indicated that the CMV gB, CMVpoly20PL-NH and CpG 1018 vaccine formulation induced significantly higher IFN-γ producing CMV-specific CD8+T cell responses compared to vaccination with CpG 1018 alone and these robust immune responses are maintained at all time points. Interestingly, there was a substantial increase in IFN-γ producing CMV-specific CD8+ T cell with each subsequent vaccination on day 28, 49 and 217 compared to other time points (FIG. 11A). A third booster dose on day 210 further produced a prompt rise in IFN-γ producing CMV-specific CD8+ T cell responses. A Similar trend (except D133) in IFN-γ producing CMV-specific CD8+ T cell was observed following in vitro stimulation of splenocytes with CMV HLA A24 restricted peptides, specifically compared to ex vivo at least a 20-fold increase in IFN-γ producing CMV-specific CD8+ T cell were observed (FIG. 11B). Interestingly, following ex vivo and in vitro stimulation of splenocytes with CMV peptides revealed that at all the time points CMV-specific CD8+ T cells demonstrated their ability to secrete multiple cytokines (FIG. 11C). A larger proportion of cells were able to secrete IFN-γ (red arc) and TNF (green arc) with the exception of day 42 ex vivo and in vitro expansion. Furthermore, from day 49 to 217 considerable population of ex vivo CMV-specific CD8+ T cells also demonstrated their ability to secrete three cytokines, IFN-γ (red arc), TNF (green arc) and IL-2 (blue arc).

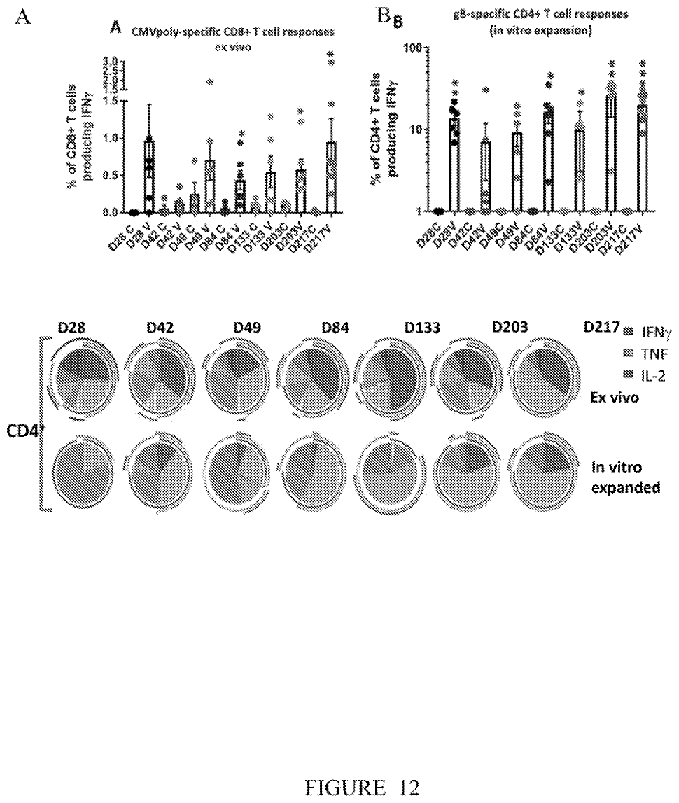

Additionally, the CMV gB, CMVpoly20PL-NH and CpG 1018 vaccine formulation also induced significant frequencies of IFN-γ producing CMV gB-specific CD4+ T cells compared to CpG 1018 alone immunisation. There was a substantial increase in ex vivo IFN-γ producing CMV gB-specific CD4+ T cell with each consequent vaccination on day 28, 49 and 84 and responses slightly declined and plateaued on day 133 and 203, nevertheless after third booster there was a rapid increase in IFN-γ producing CMV gB-specific CD4+ T cell responses were observed (FIG. 12A). Furthermore, despite low frequencies of IFN-γ producing CMV gB-specific CD4+ T cells on day 133 and 203, following in vitro stimulation of splenocytes with gB pepmix induced a greater expansion of IFN-γ producing CMV gB-specific CD4+ T cell responses at all the time points (FIG. 12B).

In addition to IFN-γ, CMV-specific CD4+ T cells also demonstrated their ability to secrete multiple cytokines (IFN-γ, TNF and IL-2) after ex vivo and in vitro expansion (FIG. 12C). In particular, from day 28 to 217 a large proportion CMV gB-specific CD4+ T cells after ex vivo stimulation with gB pepmix were capable producing three cytokine IFN-γ (red arc), TNF (green arc) and IL-2 (blue arc) or, IFN-γ (red arc) and TNF (green arc). Following in vitro expansion CMV gB-specific CD4+ T cells predominantly secreted IFN-γ and TNF (day 28, 42, 49, 84,203 and 217) or TNF and IL-2 (day 133). Taken together, these results suggest that CMV vaccine formulated with gB, CMVpoly20PL-NH and CpG1018 was capable of inducing durable CMV-specific CD4+ and CD8+ T cell responses and high frequency of these cells were able to secrete multiple cytokines simultaneously.

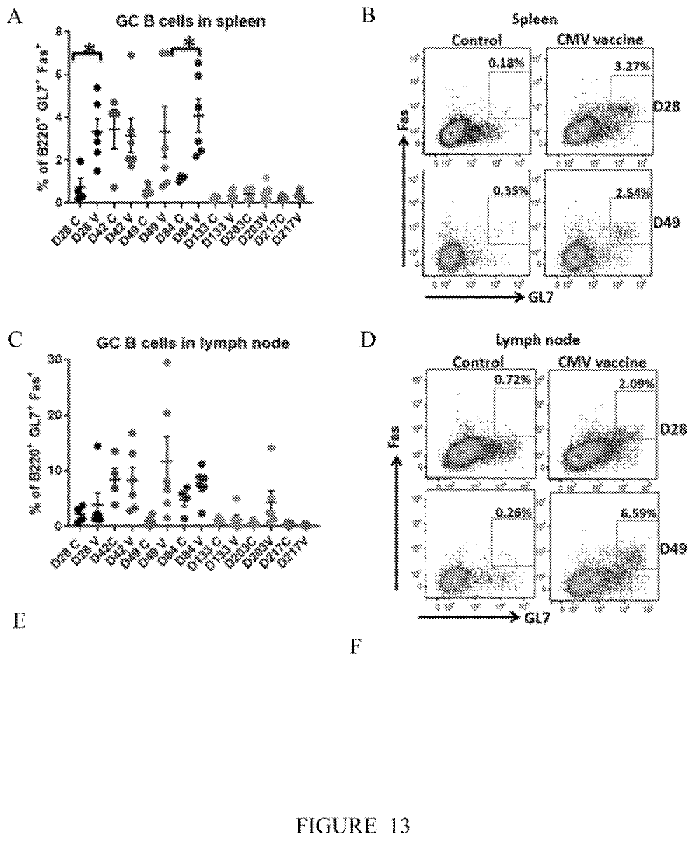

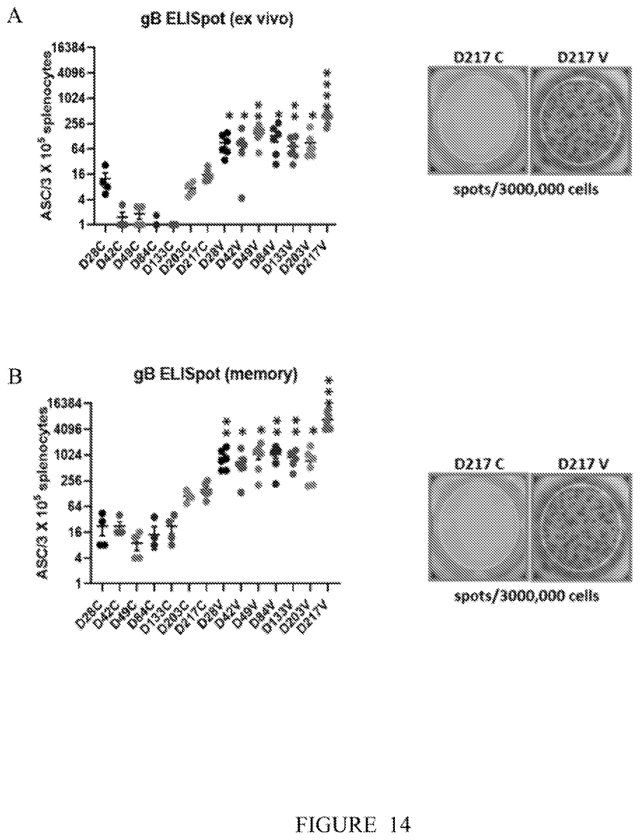

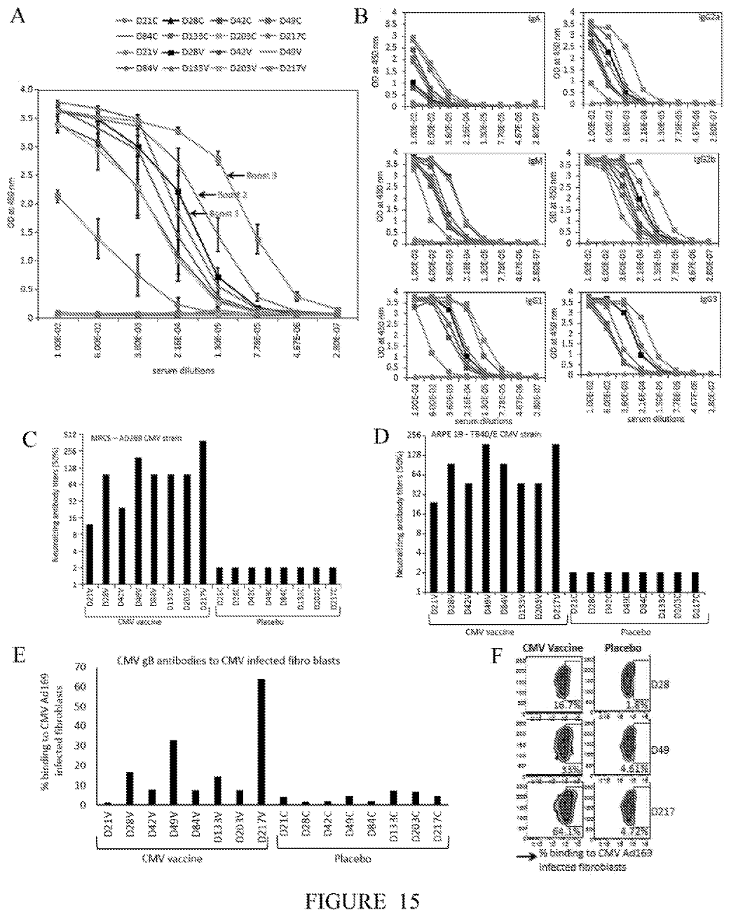

Germinal center (GCs) plays a vital role in induction of memory B cells and long-lived plasma cell to secrete the high-affinity antibodies required for long-term serological immunity (McHeyzer-Williams and McHeyzer-Williams 2005). Therefore, we verified the ability of the CMV gB, CMVpoly20PL-NH and CpG1018 vaccine formulation to induce increased germinal centre B cell response in the spleens of vaccinated and control mice. We found that the in the spleens of mice vaccinated with the CMV gB, CMVpoly20PL-NH and CpG1018 vaccine formulation induced significantly higher proportion of germinal centre B cells (B220⁺GL7⁺Fas⁺) on day 28, 49 and 84 and then declined and plateaued thereafter (FIGS. 13A and 13B); however, lymph node of vaccinated mice with the CMV gB, CMVpoly20PL-NH and CpG1018 vaccine formulation showed a modest increase on day 49 and 84 (FIGS. 13C and 13D). Further, assessment of CMV gB-specific IgG secreting plasma cells by ELISpot assay indicated that the CMV gB, CMVpoly20PL-NH and CpG1018 vaccine formulation induced a significantly higher and sustainable plasma B cell response compared to CpG 1018 alone immunisation. We also assessed the long-term memory B cell responses by ex vivo polyclonal stimulation of resting B cells and found profound increase and sustained gB-specific IgG secreting B cell responses compared to ex vivo. Interestingly, ex vivo and memory B cell analysis revealed that third immunisation resulted a rapid enhancement in the number of IgG secreting gB-specific B cells (FIGS. 14A and 14B). Additionally, to confirm whether the CMV gB, CMVpoly20PL-NH and CpG1018 vaccine formulation is also able to induce strong antibody response against gB protein, we tested serum samples from vaccinated and control mice. Serum from mice immunised with the CMV gB, CMVpoly20PL-NH and CpG1018 vaccine formulation showed significantly higher CMV gB-specific antibody titres compared to mice immunised with CpG 1018 alone and antibody titres increased with every booster dose on day 28 and 49 but showed a modest decline on day 203. However, gB-specific antibody titres peaked after third boost (FIG. 15A).

In addition, further characterisation of antibody responses revealed that CMV vaccine formulated with gB, CMVpoly20PL-NH and CpG 1018 can induce durable gB-specific multiple antibody Th1 and Th2 like Ig isotypes (IgA, IgM, IgG1, IgG2a, IgG2b and IgG3). Further enhancement of antibody titres were observed after first booster on day 28, second booster on day 49 and third booster on day 217 (FIG. 15B). Although antibody titres were lower at the time points on day 21, 42, 84, 133 and 203, CMV gB-specific antibody concentration was maintained at the detectable levels throughout the study. We further characterised these antibodies in microneutralisation assay to determine their ability to neutralise against CMV AD 169 strain infection of fibroblasts and CMV TB40/E strain infection of ARPE-19 cells. Compared to placebo immunisation with CMV vaccine induced strong neutralising antibody response against CMV AD169 and TB40/E strains. Similar to ELISA antibody titres, neutralising antibody titres were higher after the first booster on day 28, second booster on day 49 and third booster on day 217 doses. Finally, in a recent publication it has been shown that serum IgG binding to cell-associated gB is an immune correlate of vaccine efficacy (see, Jenks et al., Sci. Transl. Med. 12, eabb3611 (2020)). Therefore, in subsequent analysis we determine the ability of mouse serum antibodies binding to CMV AD169 infected fibroblasts. We found that serum antibodies from mice immunised with CMV vaccine formulated with gB, CMVpoly20PL-NH and CpG 1018 at all the time points exhibited strong binding to cell-associated gB on fibroblasts infected with CMV AD 169 strain compared to placebo. However, the strength of the binding was higher following booster doses on 28, 49 and 217 compared to day 21, 42, 84 and 133. Collectively, these data indicate that CMV vaccine formulated with gB, CMVpoly20PL-NH and CpG 1018 demonstrated its potential to induce durable and qualitative CMV-specific humoral and cellular immune responses.

### Confirmation of Long-Term Durable CMV gB, CMVpoly20PL-NH and CpG 1018 Vaccine Formulation Induced Immune Responses in HLA A1 Transgenic Mice

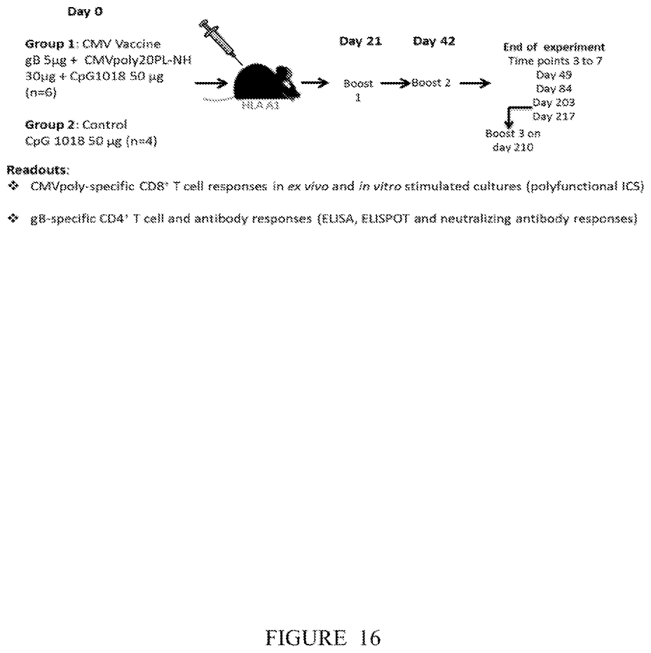

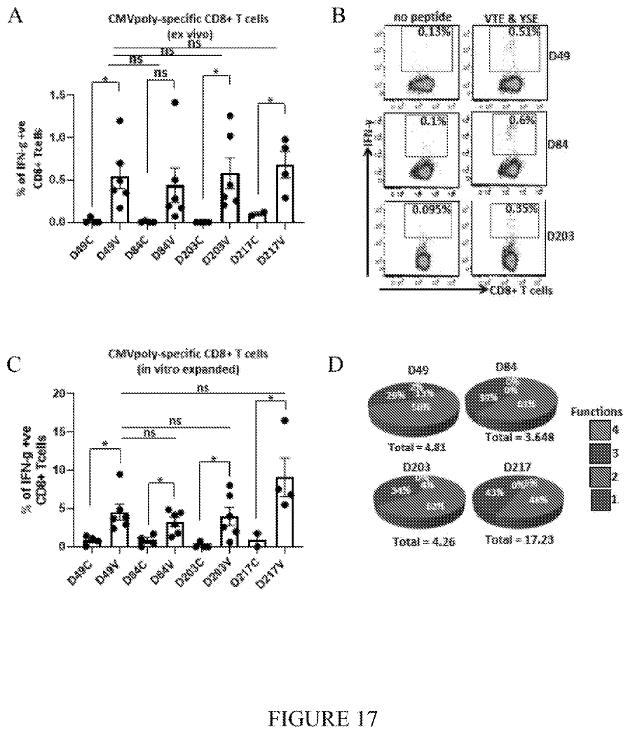

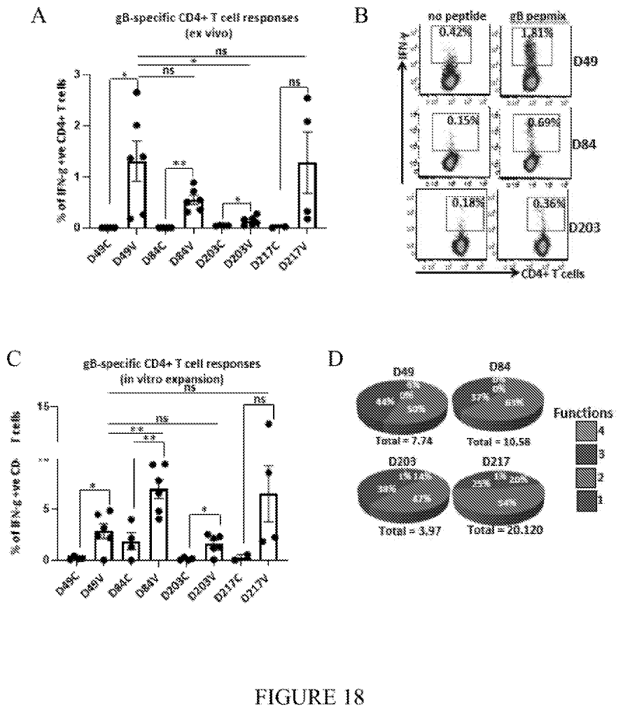

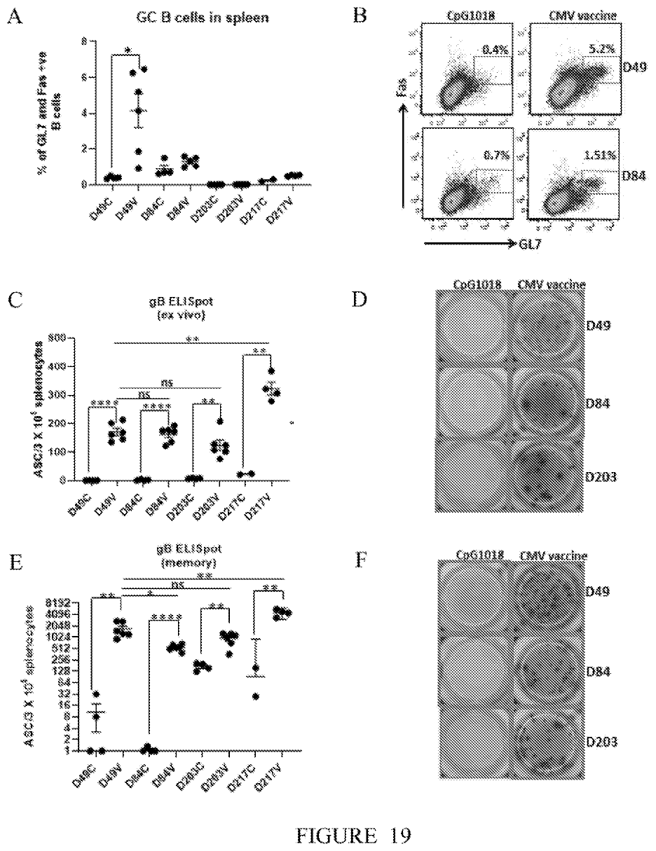

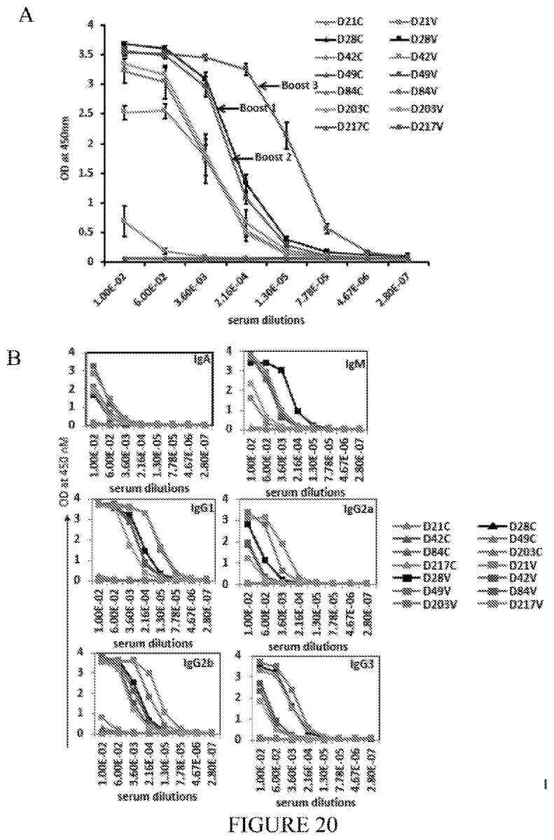

In addition to human HLA A24 transgenic mice, we also tested the long-term durability of CMV gB, CMVpoly20PL-NH and CpG1018 vaccine formulation induced immune responses in human HLA A1 transgenic mice. Human HLA A1 transgenic mice were immunised with the CMV gB, CMVpoly20PL-NH and CpG1018 vaccine formulation (vaccine group) or CpG 1018 alone (control group) on day 21, boosted on day 28, 42 and 210 and monitored CMV-specific immune responses in vaccinated human HLA A1 transgenic mice by longitudinally (on days 49, 84, 203 and 217) measuring CMVpoly20PL-NH-specific CD4+ T cell responses, gB-specific CD4+ and CD8+ T cell, B cell and antibody responses (FIG. 16). Similar to results observed in human HLA A24 transgenic mice, the CMV gB, CMVpoly20PL-NH and CpG1018 vaccine formulation induced significantly higher and sustainable frequencies of IFN-γ producing CMV-specific CD8+ T cells compared to mice immunised with CpG 1018 alone (FIGS. 17A and 17B) and there was a profound increase in IFN-γ producing CMV-specific CD8+ T cells after in vitro stimulation and expansion of vaccine induced antigen specific cells (FIG. 17C). Interestingly, CMV-specific CD8+ T cell secreted multiple cytokines simultaneously and a large proportion of cells were able to secrete 2 cytokines (IFN-γ and TNF) (FIG. 17 D). In addition, we also observed higher frequencies of IFN-γ producing CMV gB-specific CD4+ T cell responses after ex vivo (FIGS. 18A and 18B) and in vitro stimulation (FIG. 18C). Following in vitro expansion, a high proportion of CMV gB-specific CD4+T also showed their capacity to secrete two cytokines (IFN-γ and TNF) simultaneously (FIG. 18D). Further assessment of germinal centre B cell and antigen-specific IgG-secreting plasma B cell analysis indicated that the CMV gB, CMVpoly20PL-NH and CpG1018 vaccine formulation induced significantly higher proportion of germinal centre B cells (B220⁺GL7⁺Fas⁺) on day 49 and significantly higher and sustainable plasma and memory B cell responses compared to mice immunised with CpG 1018 alone (FIG. 19A-19F). Finally, to confirm the capability of CMV gB, CMVpoly20PL-NH and CpG 1018 formulation to induce gB-specific antibody responses, we tested serum samples from vaccinated and control mice. Serum from mice immunised with CMV gB, CMVpoly20PL-NH and CpG 1018 showed significantly higher gB-specific antibody titres compared to control mice and there was clearly an increasing trend in antibody titres after booster dose on day 28, 49 and 217 (FIG. 20A). The CMV gB, CMVpoly20PL-NH and CpG 1018 vaccine also induced multiple antibody Th1 like (IgG2a, IgG2b and IgG3) and Th2 like (IgM and IgG1) isotypes (FIG. 20B).

Collectively, these results indicate that a novel multivalent CMV vaccine formulation consisting of CMV gB and CMVpoly20PL-NH recombinant proteins and CpG 1018 adjuvant can induce robust CMV-specific CD4+, CD8+ and neutralising antibody responses in multiple human HLA transgenic mice. Because this vaccine is formulation is developed based on recombinant proteins and human compatible adjuvants to overcome safety issues associated with whole virus or recombinant viral vectors and improve the durability of vaccine induced immune response, it will have broader clinical implications in both prophylactic and therapeutic clinical settings, such as congenital CMV infection, transplantation and glioblastoma. The CMV gB, CMVpoly20PL-NH and CpG 1018 is highly immunogenic in multiple human HLA transgenic mice and a most notable feature of the immune response generated by this CMV vaccine is the induction of long-lived germinal centre B cells and long-term sustainable CMV-specific CD4+, CD8+ T cells, antibody secreting B cell responses and neutralising antibody responses. Interestingly, there was a gradual increase in induction of CMV-specific CD4+, CD8+ and B cell responses following every booster immunisation. In addition, antigens (gB, pp65, IE1, pp50, pp150 and DNAse) targeted in this vaccine formulation play a crucial role in all stages of CMV infection including infection, replication and packaging. These antigens are vastly recognised in the majority of CMV seropositive healthy individuals and transplant patients and known to play an important role in developing the protective immunity. All these properties make the CMV gB, CMVpoly20PL-NH and CpG 1018 vaccine formulation a superior vaccine candidate compared to existing formulations.

### REFERENCES

Adler, S. P., N. Lewis, A. Conlon, M. P. Christiansen, M. Al-Ibrahim, R. Rupp, T. M. Fu, O. Bautista, H. Tang, D. Wang, A. Fisher, T. Culp, R. Das, K. Beck, G. Tamms, L. Musey and V. S. Group (2019). “Phase 1 Clinical Trial of a Conditionally Replication-Defective Human Cytomegalovirus (CMV) Vaccine in CMV-Seronegative Subjects.” J Infect Dis 220(3): 411-419.
Amanna, I. J., N. E. Carlson and M. K. Slifka (2007). “Duration of humoral immunity to common viral and vaccine antigens.” N Engl J Med 357(19): 1903-1915.
Bernstein, D. I., E. A. Reap, K. Katen, A. Watson, K. Smith, P. Norberg, R. A. Olmsted, A. Hoeper, J. Morris, S. Negri, M. F. Maughan and J. D. Chulay (2009). “Randomized, double-blind, Phase 1 trial of an alphavirus replicon vaccine for cytomegalovirus in CMV seronegative adult volunteers.” Vaccine 28(2): 484-493.
Crough, T., L. Beagley, C. Smith, L. Jones, D. G. Walker and R. Khanna (2012). “Ex vivo functional analysis, expansion and adoptive transfer of cytomegalovirus-specific T-cells in patients with glioblastoma multiforme.” Immunol Cell Biol 90(9): 872-880.
Dasari, V., C. Smith, A. Schuessler, J. Zhong and R. Khanna (2014). “Induction of innate immune signatures following polyepitope protein-glycoprotein B-TLR4&9 agonist immunization generates multifunctional CMV-specific cellular and humoral immunity.” Hum Vaccin Immunother 10(4): 1064-1077.
Drulak, M. W., F. J. Malinoski, S. A. Fuller, S. S. Stewart, S. Hoskin, A. M. Duliege, R. Sekulovich, R. Burke and S. Winston (2000). “Vaccination of seropositive subjects with CHIRON CMV gB subunit vaccine combined with MF59 adjuvant for production of CMV immune globulin.” Viral Immunol 13(1): 49-56.
Elkington, R., N. H. Shoukry, S. Walker, T. Crough, C. Fazou, A. Kaur, C. M. Walker and R. Khanna (2004). “Cross-reactive recognition of human and primate cytomegalovirus sequences by human CD4 cytotoxic T lymphocytes specific for glycoprotein B and H.” Eur J Immunol 34(11): 3216-3226.
Elkington, R., S. Walker, T. Crough, M. Menzies, J. Tellam, M. Bharadwaj and R. Khanna (2003). “Ex vivo profiling of CD8+-T-cell responses to human cytomegalovirus reveals broad and multispecific reactivities in healthy virus carriers.” J Virol 77(9): 5226-5240. Gibson, L., C. M. Barysauskas, M. McManus, S. Dooley, D. Lilleri, D. Fisher, T. Srivastava, D. J. Diamond and K. Luzuriaga (2015). “Reduced frequencies of polyfunctional CMV-specific T cell responses in infants with congenital CMV infection.” J Clin Immunol 35(3): 289-301.
Jacobson, M. A., E. Sinclair, B. Bredt, L. Agrillo, D. Black, C. L. Epling, A. Carvidi, T. Ho, R. Bains, V. Girling and S. P. Adler (2006). “Safety and immunogenicity of Towne cytomegalovirus vaccine with or without adjuvant recombinant interleukin-12.” Vaccine 24(25): 5311-5319.
La Rosa, C., J. Longmate, J. Martinez, Q. Zhou, T. I. Kaltcheva, W. Tsai, J. Drake, M. Carroll, F. Wussow, F. Chiuppesi, N. Hardwick, S. Dadwal, I. Aldoss, R. Nakamura, J. A. Zaia and D. J. Diamond (2017). “MVA vaccine encoding CMV antigens safely induces durable expansion of CMV-specific T cells in healthy adults.” Blood 129(1): 114-125. Lee, S. W., P. F. Markham, M. J. Coppo, A. R. Legione, J. F. Markham, A. H. Noormohammadi, G. F. Browning, N. Ficorilli, C. A. Hartley and J. M. Devlin (2012). “Attenuated vaccines can recombine to form virulent field viruses.” Science 337(6091): 188.
Manley, T. J., L. Luy, T. Jones, M. Boeckh, H. Mutimer and S. R. Riddell (2004). “Immune evasion proteins of human cytomegalovirus do not prevent a diverse CD8+ cytotoxic T-cell response in natural infection.” Blood 104(4): 1075-1082.
McHeyzer-Williams, L. J. and M. G. McHeyzer-Williams (2005). “Antigen-specific memory B cell development.” Annu Rev Immunol 23: 487-513.
Pepperl-Klindworth, S., N. Frankenberg and B. Plachter (2002). “Development of novel vaccine strategies against human cytomegalovirus infection based on subviral particles.” J Clin Virol 25 Suppl 2: S75-85.
Sylwester, A. W., B. L. Mitchell, J. B. Edgar, C. Taormina, C. Pelte, F. Ruchti, P. R. Sleath, K. H. Grabstein, N. A. Hosken, F. Kern, J. A. Nelson and L. J. Picker (2005). “Broadly targeted human cytomegalovirus-specific CD4+ and CD8+ T cells dominate the memory compartments of exposed subjects.” J Exp Med 202(5): 673-685.
Wloch, M. K., L. R. Smith, S. Boutsaboualoy, L. Reyes, C. Han, J. Kehler, H. D. Smith, L. Selk, R. Nakamura, J. M. Brown, T. Marbury, A. Wald, A. Rolland, D. Kaslow, T. Evans and M. Boeckh (2008). “Safety and immunogenicity of a bivalent cytomegalovirus DNA vaccine in healthy adult subjects.” J Infect Dis 197(12): 1634-1642.
Zhong, J. and R. Khanna (2009). “Ad-gBCMVpoly: A novel chimeric vaccine strategy for human cytomegalovirus-associated diseases.” J Clin Virol.

Throughout the specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the scope of the present invention.

All computer programs, algorithms, patent and scientific literature referred to herein is incorporated herein by reference in their entirety.

1. A pharmaceutical composition comprising:
(a) one or a plurality of isolated proteins comprising a plurality of epitopes, wherein the plurality of epitopes are derived from two or more different CMV antigens;
(b) a CMV envelope protein, or a fragment, variant or derivative thereof; and
(c) a TLR9 agonist.

2. The pharmaceutical composition of claim 1, wherein the composition is capable of inducing or eliciting a humoral immune response and a cell-mediated immune response, such as a cytotoxic T-lymphocyte immune response, upon administration to a subject.

3. The pharmaceutical composition of claim 1, wherein the one or plurality of isolated proteins is or comprises a polytope protein comprising two or more of the plurality of epitopes from the two or more different CMV antigens.

4. The pharmaceutical composition of claim 3, wherein the polytope protein comprises an intervening amino acid sequence between at least two of said epitopes, wherein the intervening amino acid sequence comprises a proteasome liberation amino acid sequence.

5. The pharmaceutical composition of claim 4, wherein the proteasome liberation amino acids or amino acid sequences comprise AD, K and/or R.

6. The pharmaceutical composition of any one of the preceding claims, wherein the epitopes are restricted by HLA class I specificities HLA-A1, -A2, -A3, -A11, -A23, -A24, -A26, -A29, -A30, -B7, -B8, -B18, -B27, -B35, -B38, -B40, -B41, -B44, -B51, -B57, -B58 and/or -CW6.

7. The pharmaceutical composition of any one of the preceding claims, wherein the epitopes are derived from pp50, pp65, pp150, DNAse and/or IE-1.

8. The pharmaceutical composition of claim 7, wherein the epitopes have an amino acid sequence selected from the group consisting of the amino acid sequences set forth in SEQ ID NOS: 1-20, Table 1, a fragment, variant or derivative thereof and any combination thereof.

9. The pharmaceutical composition of claim 8, wherein the one or plurality of isolated proteins comprise each of the epitope amino acid sequences set forth in SEQ ID NOS: 1-20.

10. The pharmaceutical composition of any one of the preceding claims, wherein the one or plurality of isolated proteins comprise an amino acid sequence set forth in SEQ ID NO:21 or a fragment, variant or derivative thereof.

11. The pharmaceutical composition of any one of the preceding claims, wherein the one or plurality of isolated proteins comprise twenty (20) or less epitopes.

12. The pharmaceutical composition of any one of the preceding claims, further comprising a pharmaceutically-acceptable carrier, diluent or excipient.

13. The pharmaceutical composition of any one of the preceding claims, wherein the CMV envelope protein is or comprises CMV glycoprotein B, or a fragment, variant or derivative thereof.

14. The pharmaceutical composition of any one of the preceding claims, wherein the TLR9 agonist is or comprises CpG ODN1018 and/or CpG ODN2006.

15. A vaccine that comprises the pharmaceutical composition of any one of the preceding claims for eliciting a protective immune response against CMV.

16. A method of treating or preventing a CMV infection in a subject, said method including the step of administering to the subject a therapeutically effective amount of a pharmaceutical composition comprising:
(a) one or a plurality of isolated proteins comprising a plurality of epitopes, wherein the plurality of epitopes are derived from two or more different CMV antigens;
(b) a CMV envelope protein, or a fragment, variant or derivative thereof; and
(c) a TLR9 agonist;
to thereby prevent or treat the CMV infection in the subject.

17. A method of eliciting an immune response to a CMV antigen in a subject, said method including the step of administering to the subject a therapeutically effective amount of a pharmaceutical composition comprising:
(a) one or a plurality of isolated proteins comprising a plurality of epitopes, wherein the plurality of epitopes are derived from two or more different CMV antigens;
(b) a CMV envelope protein, or a fragment, variant or derivative thereof; and
(c) a TLR9 agonist;
to thereby elicit the immune response in said subject.

18. The method of claim 17, wherein the immune response is or comprises a humoral immune response and a cell-mediated immune response, such as a cytotoxic T-lymphocyte immune response.

19. The method of claim 17 or claim 18, which elicits a protective immune response against CMV or a CMV infection in the subject.

20. A method of inducing immunity against a CMV infection in a subject, said method including the step of administering to the subject a therapeutically effective amount of a pharmaceutical composition comprising:
(a) one or a plurality of isolated proteins comprising a plurality of epitopes, wherein the plurality of epitopes are derived from two or more different CMV antigens;
(b) a CMV envelope protein, or a fragment, variant or derivative thereof; and
(c) a TLR9 agonist;
to thereby induce immunity against the CMV infection in the subject.

21. The method of any one of claims 16 to 20, wherein the subject is a human.

22. The method of any one of claims 16 to 21, wherein the pharmaceutical composition is that of any one of claims 1 to 14.

23. An isolated protein comprising each of the epitope amino acid sequences set forth in SEQ ID NOS: 1-20, or fragments, variants or derivatives thereof.

24. The isolated protein of claim 23, wherein the isolated protein comprises the amino acid sequence set forth in SEQ ID NO:21 or a fragment, variant or derivative thereof.

25. An isolated nucleic acid encoding the isolated protein of claim 23 or claim 24.
